(19) United States
(12) Patent Application Publication
(10) Pub. No.: US 2011/0223201 A1
(43) Pub. Date: Sep. 15, 2011

(54) Immunonanotherapeutics Providing a Th1-Biased Response

(75) Inventors: Grayson B. Lipford, Watertown, MA (US); Robert L. Bratzler, Concord, MA (US)

(73) Assignee: Selecta Biosciences, Inc., Watertown, MA (US)

(21) Appl. No.: 12/764,569

(22) Filed: Apr. 21, 2010

Related U.S. Application Data
(60) Provisional application No. 61/214,229, filed on Apr. 21, 2009.

(51) Int. Cl. A61K 9/14; A61K 39/39; A61P 37/04

(52) U.S. Cl. 424/400; 424/193.1

(57) ABSTRACT

Disclosed are synthetic nanocarrier compositions, and related methods, for treating diseases in which generating a Th1-biased immune response is desirable.

### RELATED APPLICATIONS

This application claims the benefit under 35 U.S.C. §119 of U.S. provisional application 61/214,229, filed Apr. 21, 2009, the contents of which are incorporated herein by reference in their entirety.

### FIELD OF THE INVENTION

This invention relates to synthetic nanocarrier compositions, and related methods, for treating diseases in which generating a Th1-biased immune response is desirable.

### BACKGROUND OF THE INVENTION

There are many diseases where the immune system itself actually appears to play a significant role in mediating the disease. This can occur when an immune stimulus causes activated CD4 T cells to differentiate into Th2 cells which then secrete Th2-associated cytokines, such as interleukin (IL)-4, IL-5, IL-10, and IL-13. B cells that are stimulated in the presence of Th2 cytokines respond by preferentially producing certain antibody isotypes, particularly IgE. IgE-dependent immune responses to certain antigens and the action of Th2 cytokines can cause clinical symptoms associated with atopic conditions such as allergies, asthma, and atopic dermatitis. Additionally, in certain conditions such as certain chronic infectious diseases and cancer, an amplified Th1 response is desired to effect a better outcome for the conditions.

While some treatments for conditions characterized by an undesirable Th2 biased immune response are known, improved therapies are needed. Further, improved therapies for diseases in which Th1-biased responses of a subject's immune system are suboptimal or ineffective are also needed.

Accordingly, improved compositions and related methods are needed to provide improved therapies for Th2-mediated diseases and for diseases in which an enhanced Th1-biased response of a subject's immune system is desirable.

### SUMMARY OF THE INVENTION

In an aspect, the invention relates to a composition for treatment of a condition comprising: synthetic nanocarriers comprising (1) an immunofeature surface, and (2) a Th1 biasing immunostimulatory agent coupled to the synthetic nanocarriers; and a pharmaceutically acceptable excipient; wherein the immunofeature surface does not comprise antigen that is relevant to treatment of the condition in an amount sufficient to provoke an adaptive immune response to the antigen that is relevant to treatment of the condition.

In another aspect, the invention relates to a method comprising: identifying a subject suffering from a condition; providing a composition that comprises synthetic nanocarriers that comprise (1) an APC targeting feature, and (2) a Th1 biasing immunostimulatory agent coupled to the synthetic nanocarriers; and a pharmaceutically acceptable excipient; and administering the composition to the subject; wherein the administration of the composition does not further comprise co-administration of an antigen that is relevant to treatment of the condition.

In yet another aspect, the invention relates to a method comprising: providing a composition comprising synthetic nanocarriers that comprise a Th1 biasing immunostimulatory agent and an APC targeting feature; administering the composition to a subject; and administering an antigen to the subject to which a Th1 biased response is desired at a time different from administration of the composition to the subject; wherein administration of the antigen comprises passive administration or active administration.

### BRIEF DESCRIPTION OF THE DRAWINGS

FIG. 1 shows BALF eosinophil differential cell counts (% of total cells). Differential cell counts were done on cytospins of BALF 48 hours after the last ovalbumin challenge. Ovalbumin sensitized mice were treated with CpG i.p. (1), nanocarriers with R848 i.p. (2), or nanocarriers with R848 i.n. (3) 24 hours before each ovalbumin challenge. Data represent mean±SD of 5 mice per treatment group. Treatment groups labeled as sensitization (PBS, OVA, or OVA+alum), treatment (PBS, CpG, or nanocarriers±R848), and challenge (PBS or OVA).

FIG. 2A shows Cytokines in BALF at 18 hours after final ovalbumin challenge. IL-4 levels (pg/mL) were measured by ELISA. Ovalbumin sensitized mice were treated with CpG i.p. (1), nanocarriers with R848 i.p. (2), nanocarriers with R848 i.n. (3), or R848 i.p. (4) 24 hours before each ovalbumin challenge. Data represent mean±SD of 5 mice per treatment group. Treatment groups labeled as sensitization (PBS, OVA, or OVA+alum), treatment (PBS, CpG, or nanocarriers±R848), and challenge (PBS or OVA).

FIG. 2B shows Cytokines in BALF at 18 hours after final ovalbumin challenge. IL-5 levels (pg/mL) were measured by ELISA. Ovalbumin sensitized mice were treated with CpG i.p. (1), nanocarriers with R848 i.p. (2), nanocarriers with R848 i.n. (3), or R848 i.p. (4) 24 hours before each ovalbumin challenge. Data represent mean±SD of 5 mice per treatment group. Treatment groups labeled as sensitization (PBS, OVA, or OVA+alum), treatment (PBS, CpG, or nanocarriers±R848), and challenge (PBS or OVA).

FIG. 2C shows Cytokines in BALF at 18 hours after final ovalbumin challenge. IL-13 levels (pg/mL) were measured by ELISA. Ovalbumin sensitized mice were treated with CpG i.p. (1), nanocarriers with R848 i.p. (2), nanocarriers with R848 i.n. (3), or R848 i.p. (4) 24 hours before each ovalbumin challenge. Data represent mean±SD of 5 mice per treatment group. Treatment groups labeled as sensitization (PBS, OVA, or OVA+alum), treatment (PBS, CpG, or nanocarriers±R848), and challenge (PBS or OVA).

FIG. 2D shows Cytokines in BALF at 18 hours after final ovalbumin challenge. IL-12p40 levels (pg/mL) were measured by ELISA. Ovalbumin sensitized mice were treated with CpG i.p. (1), nanocarriers with R848 i.p. (2), nanocarriers with R848 i.n. (3), or R848 i.p. (4) 24 hours before each ovalbumin challenge. Data represent mean±SD of 5 mice per treatment group. Treatment groups labeled as sensitization (PBS, OVA, or OVA+alum), treatment (PBS, CpG, or nanocarriers±R848), and challenge (PBS or OVA).

### DETAILED DESCRIPTION OF THE INVENTION

Before describing the present invention in detail, it is to be understood that this invention is not limited to particularly exemplified materials or process parameters as such may, of course, vary. It is also to be understood that the terminology used herein is for the purpose of describing particular embodiments of the invention only, and is not intended to be limiting of the use of alternative terminology to describe the present invention.

All publications, patents and patent applications cited herein, whether supra or infra, are hereby incorporated by reference in their entirety for all purposes.

As used in this specification and the appended claims, the singular forms “a,” “an” and “the” include plural referents unless the content clearly dictates otherwise. For example, reference to “a polymer” includes a mixture of two or more such molecules, reference to “a solvent” includes a mixture of two or more such solvents, reference to “an adhesive” includes mixtures of two or more such materials, and the like.

### A. INTRODUCTION

The inventors have unexpectedly and surprisingly discovered that the problems and limitations noted above can be overcome by practicing the invention disclosed herein. In particular, the inventors have unexpectedly discovered that it is possible to provide compositions and methods that relate to a composition for treatment of a condition comprising: synthetic nanocarriers comprising (1) an immunofeature surface, and (2) a Th1 biasing immunostimulatory agent coupled to the synthetic nanocarriers; and a pharmaceutically acceptable excipient; wherein the immunofeature surface does not comprise antigen that is relevant to treatment of the condition in an amount sufficient to provoke an adaptive immune response to the antigen that is relevant to treatment of the condition.

Further, the inventors have unexpectedly discovered that it is possible to provide compositions and methods that relate to a method comprising: identifying a subject suffering from a condition; providing a composition that comprises synthetic nanocarriers that comprise (1) an APC targeting feature, and (2) a Th1 biasing immunostimulatory agent coupled to the synthetic nanocarriers; and a pharmaceutically acceptable excipient; and administering the composition to the subject; wherein the administration of the composition does not further comprise co-administration of an antigen that is relevant to treatment of the condition.

Additionally, the inventors have unexpectedly discovered that it is possible to provide compositions and methods that relate to a method comprising: providing a composition comprising synthetic nanocarriers that comprise a Th1 biasing immunostimulatory agent and an APC targeting feature; administering the composition to a subject; and administering an antigen to the subject to which a Th1 biased response is desired at a time different from administration of the composition to the subject; wherein administration of the antigen comprises passive administration or active administration.

One approach to prevent or treat diseases that are characterized by an undesirable Th2-biased response, or a suboptimal/ineffective Th1 response, are immunological interventions that counteract the differentiation of Th2 cells and the action of Th2 cytokines. This can be achieved by exposing the body to conditions that result in the production of Th1 cells and Th1-associated cytokines, including interferon-gamma, IL-12 and IL-18. Such conditions are referred to as a “Th1 biased response.” Dendritic cells are thought to play an important role in both the induction and maintenance of allergic diseases and also in the treatment-induced switching to a Th1 response. Thus, treatments directed at dendritic cells that boost the capacity of dendritic cells to promote Th1 responses represent a promising avenue for a mechanism-based treatment of allergy and asthma.

In the present invention, the inventors have unexpectedly discovered that certain types of immunonanotherapeutics can be utilized to induce a Th1 biased response under conditions that would normally generate either a Th2 biased response or a suboptimal/ineffective Th1 biased response. This is accomplished through the use of compositions comprising immunonanotherapeutics that (1) are targeted to antigen presenting cells using APC targeting features, and (2) do not comprise antigen that is relevant to treatment of the condition. Instead, the antigen is not co-administered; rather it is administered to a subject separately usually at a time different than administration of an inventive composition. In certain related embodiments, the antigen might be administered either actively or passively.

The Th1 biased state following administration of an inventive composition generally lasts for a period of time long enough for the antigen that is relevant to treatment of the condition to be administered to the subject, either actively or passively. In embodiments, the Th1 biased state may be long lasting, regardless of whether or not the antigen is administered actively or passively.

Examples 1-7 detail several different specific embodiments of the invention, including inventive nanocarriers, and applications thereof. Example 8 details the use of an embodiment of the present invention in the treatment of experimental asthma.

The present invention will now be described in more detail.

### B. DEFINITIONS

“Active administration” means the administration of a substance, such as an antigen, by directly administering the substance to the subject or taking a positive action that results in the subject's exposure to the substance. For instance, injecting, or orally dosing, an allergen or a chronic infectious agent antigen to the subject are embodiments of active administration. In another embodiment, inducing tumor cell death in a subject in a manner that results in the generation of tumor antigens to which a subject is exposed is an embodiment of active administration.

“Administering” or “administration” means (1) dosing a pharmacologically active material, such as an inventive composition, to a subject in a manner that is pharmacologically useful, (2) directing that such material be dosed to the subject in a pharmacologically useful manner, or (3) directing the subject to self-dose such material in a pharmacologically useful manner.

“Allergen” means a substance that triggers an immediate hypersensitivity reaction, characterized by binding to allergen-specific IgE and activation of IgE receptor bearing cells resulting in a Th2-type pattern of cytokine response as well as histamine release. Included in such immediate hypersensitivity reactions are indications such as allergy and allergic asthma. In an embodiment, immunofeature surfaces according to the invention do not comprise an allergen.

“Antigen that is relevant to treatment of the condition” means an antigen to which an adaptive immune response (as distinguished, for example, from an innate immune response) would treat or alleviate a particular condition in a subject following administration of the antigen to the subject. In an embodiment, immunofeature surfaces according to the invention do not comprise an antigen that is relevant to treatment of the condition. In an embodiment, administration of the composition does not further comprise administration of an antigen that is relevant to treatment of the condition, wherein the antigen may be either coupled to the nanocarriers or not coupled to the nanocarriers. In an embodiment, the antigen that is relevant to treatment of the condition is administered at a time different from a time when the composition is administered. In embodiments, the condition being treated does not need to be specified, since the requirement is that the antigen is known or expected to be relevant to treatment of the condition.

“Antigen to the subject to which a Th1 biased response is clinically beneficial” means an antigen that would typically elicit a Th2-type cytokine response from a subject, but to which a bias towards a response that is characterized by a Th1-type cytokine response would be useful clinically. In an embodiment, an antigen to the subject to which a Th1 biased response is clinically beneficial is administered to a subject at a time different from administration of the composition.

“APC targeting feature” means one or more portions of which the inventive synthetic nanocarriers are comprised that target the synthetic nanocarriers to professional antigen presenting cells (“APCs”), such as but not limited to dendritic cells, SCS macrophages, follicular dendritic cells, and B cells. In embodiments, APC targeting features may comprise immunofeature surface(s) and/or targeting moieties that bind known targets on APCs.

In embodiments, targeting moieties for known targets on macrophages (“Mphs”) comprise any targeting moiety that specifically binds to any entity (e.g., protein, lipid, carbohydrate, small molecule, etc.) that is prominently expressed and/or present on macrophages (i.e., subcapsular sinus-Mph markers). Exemplary SCS-Mph markers include, but are not limited to, CD4 (L3T4, W3/25, T4); CD9 (p24, DRAP-1, MRP-1); CD11a (LFA-1α, αL Integrin chain); CD11b (αM Integrin chain, CR3, Mo1, C3niR, Mac-1); CD11c (αX Integrin, p150, 95, AXb2); CDw12 (p90-120); CD13 (APN, gp150, EC 3.4.11.2); CD14 (LPS-R); CD15 (X-Hapten, Lewis, X, SSEA-1,3-FAL); CD15s (Sialyl Lewis X); CD15u (3′ sulpho Lewis X); CD15su (6 sulpho-sialyl Lewis X); CD16a (FCRIIIA); CD16b (FcgRIIIb); CDw17 (Lactosylceramide, LacCer); CD18 (Integrin β2, CD11a,b,c β-subunit); CD26 (DPP IV ectoeneyme, ADA binding protein); CD29 (Platelet GPIIa, β-1 integrin, GP); CD31 (PECAM-1, Endocam); CD32 (FCγRII); CD33 (gp67); CD35 (CR1, C3b/C4b receptor); CD36 (GpIIIb, GPIV, PASIV); CD37 (gp52-40); CD38 (ADP-ribosyl cyclase, T10); CD39 (ATPdehydrogenase, NTPdehydrogenase-1); CD40 (Bp50); CD43 (Sialophorin, Leukosialin); CD44 (EMCRII, H-CAM, Pgp-1); CD45 (LCA, T200, B220, Ly5); CD45RA; CD45RB; CD45RC; CD45RO (UCHL-1); CD46 (MCP); CD47 (gp42, IAP, OA3, Neurophillin); CD47R (MEM-133); CD48 (Blast-1, Hulym3, BCM-1, OX-45); CD49a (VLA-1α, α1 Integrin); CD49b (VLA-2α, gpla, α2 Integrin); CD49c (VLA-3α, α3 Integrin); CD49e (VLA-5α, α5 Integrin); CD49f (VLA-6α, α6 Integrin, gplc); CD50 (ICAM-3); CD51 (Integrin α, VNR-α, Vitronectin-Rα); CD52 (CAMPATH-1, HE5); CD53 (OX-44); CD54 (ICAM-1); CD55 (DAF); CD58 (LFA-3); CD59 (1F5Ag, H19, Protectin, MACIF, MIRL, P-18); CD60a (GD3); CD60b (9-O-acetyl GD3); CD61 (GP IIIa, β3 Integrin); CD62L (L-selectin, LAM-1, LECAM-1, MEL-14, Leu8, TQ1); CD63 (LIMP, MLA1, gp55, NGA, LAMP-3, ME491); CD64 (FcγRI); CD65 (Ceramide, VIM-2); CD65s (Sialylated-CD65, VIM2); CD72 (Ly-19.2, Ly-32.2, Lyb-2); CD74 (Ii, invariant chain); CD75 (sialo-masked Lactosamine); CD75S (α2,6 sialylated Lactosamine); CD80 (B7, B7-1, BB1); CD81 (TAPA-1); CD82 (4F9, C33, IA4, KAI1, R2); CD84 (p75, GR6); CD85a (ILT5, LIR2, HL9); CD85d (ILT4, LIR2, MIR10); CD85j (ILT2, LIR1, MIR7); CD85k (ILT3, LIR5, HM18); CD86 (B7-2/B70); CD87 (uPAR); CD88 (C5aR); CD89 (IgA Fc receptor, FcαR); CD91 (α2M-R, LRP); CDw92 (p70); CDw93 (GR11); CD95 (APO-1, FAS, TNFRSF6); CD97 (BL-KDD/F12); CD98 (4F2, FRP-1, RL-388); CD99 (MIC2, E2); CD99R (CD99 Mab restricted); CD100 (SEMA4D); CD101 (IGSF2, P126, V7); CD102 (ICAM-2); CD111 (PVRL1, HveC, PRR1, Nectin 1, HIgR); CD112 (HveB, PRR2, PVRL2, Nectin2); CD114 (CSF3R, G-CSRF, HG-CSFR); CD115 (c-fms, CSF-1R, M-CSFR); CD116 (GMCSFRα); CDw119 (IFNγR, IFNγyRA); CD120a (TNFRI, p55); CD120b (TNFRII, p75, TNFR p80); CD121b (Type 2 IL-1R); CD122 (IL2Rβ); CD123 (IL-3Rα); CD124 (IL-4Rα); CD127 (p90, IL-7R, IL-7Rα); CD128a (IL-8Ra, CXCR1, (Tentatively renamed as CD181)); CD128b (IL-8Rb, CSCR2, (Tentatively renamed as CD182)); CD130 (gp130); CD131 (Common β subunit); CD132 (Common γ chain, IL-2Rγ); CDw136 (MSP-R, RON, p158-ron); CDw137 (4-1BB, ILA); CD139; CD141 (Thrombomodulin, Fetomodulin); CD147 (Basigin, EMMPRIN, M6, OX47); CD148 (HPTP-η, p260, DEP-1); CD155 (PVR); CD156a (CD156, ADAMS, MS2); CD156b (TACE, ADAM17, cSVP); CDw156C (ADAM10); CD157 (Mo5, BST-1); CD162 (PSGL-1); CD164 (MGC-24, MUC-24); CD165 (AD2, gp37); CD168 (RHAMM, IHABP, HMMR); CD169 (Sialoadhesin, Siglec-1); CD170 (Siglec 5); CD171 (L1CAM, NILE); CD172 (SIRP-1α, MyD-1); CD172b (SIRPβ); CD180 (RP105, Bgp95, Ly64); CD181 (CXCR1, (Formerly known as CD128a)); CD182 (CXCR2, (Formerly known as CD128b)); CD184 (CXCR4, NPY3R); CD191 (CCR1); CD192 (CCR2); CD195 (CCR5); CDw197 (CCR7 (was CDw197)); CDw198 (CCR8); CD204 (MSR); CD205 (DEC-25); CD206 (MMR); CD207 (Langerin); CDw210 (CK); CD213a (CK); CDw217 (CK); CD220 (Insulin R); CD221 (IGF1R); CD222 (M6P-R, IGFII-R); CD224 (GGT); CD226 (DNAM-1, PTA1); CD230 (Prion Protein (PrP)); CD232 (VESP-R); CD244 (2B4, P38, NAIL); CD245 (p220/240); CD256 (APRIL, TALL2, TNF (ligand) superfamily, member 13); CD257 (BLYS, TALL1, TNF (ligand) superfamily, member 13b); CD261 (TRAIL-R1, TNF-R superfamily, member 10a); CD262 (TRAIL-R2, TNF-R superfamily, member 10b); CD263 (TRAIL-R3, TNBF-R superfamily, member 10c); CD264 (TRAIL-R4, TNF-R superfamily, member 10d); CD265 (TRANCE-ft TNF-R superfamily, member 11a); CD277 (BT3.1, B7 family: Butyrophilin 3); CD280 (TEM22, END0180); CD281 (TLR1, TOLL-like receptor 1); CD282 (TLR2, TOLL-like receptor 2); CD284 (TLR4, TOLL-like receptor 4); CD295 (LEPR); CD298 (ATP1B3, Na K ATPase, β3 subunit); CD300a (CMRF-35H); CD300c (CMRF-35A); CD300e (CMRF-35L1); CD302 (DCL1); CD305 (LAIR1); CD312 (EMR2); CD315 (CD9P1); CD317 (BST2); CD321 (JAM1); CD322 (JAM2); CDw328 (Siglec7); CDw329 (Siglec9); CD68 (gp 110, Macrosialin); and/or mannose receptor; wherein the names listed in parentheses represent alternative names.

In embodiments, targeting moieties for known targets on dendritic cells (“DCs”) comprise any targeting moiety that specifically binds to any entity (e.g., protein, lipid, carbohydrate, small molecule, etc.) that is prominently expressed and/or present on DCs (i.e., a DC marker). Exemplary DC markers include, but are not limited to, CD1a (R4, T6, HTA-1); CD1b (R1); CD1c (M241, R7); CD1d (R3); CD1e (R2); CD11b (αM Integrin chain, CR3, Mo1, C3niR, Mac-1); CD11c (αX Integrin, p150, 95, AXb2); CDw117 (Lactosylceramide, LacCer); CD19 (B4); CD33 (gp67); CD 35 (CR1, C3b/C4b receptor); CD 36 (GpIIIb, GPIV, PASIV); CD39 (ATPdehydrogenase, NTPdehydrogenase-1); CD40 (Bp50); CD45 (LCA, T200, B220, Ly5); CD45RA; CD45RB; CD45RC; CD45RO (UCHL-1); CD49d (VLA-4α, α4 Integrin); CD49e (VLA-5α, α5 Integrin); CD58 (LFA-3); CD64 (FcγRI); CD72 (Ly-19.2, Ly-32.2, Lyb-2); CD73 (Ecto-5′ nucloticlase); CD74 (Ii, invariant chain); CD80 (B7, B7-1, BB1); CD81 (TAPA-1); CD83 (HB15); CD85a (ILT5, LIR3, HL9); CD85d (ILT4, LIR2, MIR10); CD85j (ILT2, LIR1, MIR7); CD85k (ILT3, LIR5, HM18); CD86 (B7-2/B70); CD88 (C5aB); CD97 (BL-KDD/F12); CD101 (IGSF2, P126, V7); CD116 (GM-CSFRα); CD120a (TMFR1, p55); CD120b (TNFRII, p75, TNFR p80); CD123 (IL-3Rα); CD139; CD148 (HPTP-η, DEP-1); CD150 (SLAM, IPO-3); CD156b (TACE, ADAM17, cSVP); CD157 (Mo5, BST-1); CD167a (DDR1, trkE, cak); CD168 (RHAMM, 1HABP, HMMR); CD169 (Sialoadhesin, Siglec-1); CD170 (Siglec-5); CD171 (L1CAM, NILE); CD172 (SIRP-1α, MyD-1); CD172b (SIRPβ); CD180 (RP105, Bgp95, Ly64); CD184 (CXCR4, NPY3R); CD193 (CCR3); CD196 (CCR6); CD197 (CCR7 (ws CDw197)); CDw197 (CCR7, EBI1, BLR2); CD200 (OX2); CD205 (DEC-205); CD206 (MMR); CD207 (Langerin); CD208 (DC-LAMP); CD209 (DCSIGN); CDw218a (IL18Rα); CDw218b (IL81Rα); CD227 (MUC1, PUM, PEM, EMA); CD230 (Prion Protein (PrP)); CD252 (OX40L, TNF (ligand) superfamily, member 4); CD258 (LIGHT, TNF (ligand) superfamily, member 14); CD265 (TRANCE-R, TNF-R superfamily, member 11a); CD271 (NGFR, p75, TNFR superfamily, member 16); CD273 (B7DC, PDL2); CD274 (B7H1, PDL1); CD275 (B7H2, ICOSL); CD276 (B7H3); CD277 (BT3.1, B7 family: Butyrophilin 3); CD283 (TLR3, TOLL-like receptor 3); CD289 (TLR9, TOLL-like receptor 9); CD295 (LEPR); CD298 (ATP1B3, Na K ATPase β3 submit); CD300a (CMRF-35H); CD300c (CMRF-35A); CD301 (MGL1, CLECSF14); CD302 (DCL1); CD303 (BDCA2); CD304 (BDCA4); CD312 (EMR2); CD317 (BST2); CD319 (CRACC, SLAMF7); CD320 (8D6); and CD68 (gp110, Macrosialin); class II MHC; BDCA-1; Siglec-H; wherein the names listed in parentheses represent alternative names.

In embodiments, targeting can be accomplished by any targeting moiety that specifically binds to any entity (e.g., protein, lipid, carbohydrate, small molecule, etc.) that is prominently expressed and/or present on B cells (i.e., B cell marker). Exemplary B cell markers include, but are not limited to, CD1c (M241, R7); CD1d (R3); CD2 (E-rosette R, T11, LFA-2); CD5 (T1, Tp67, Leu-1, Ly-1); CD6 (T12); CD9 (p24, DRAP-1, MRP-1); CD11a (LFA-1α, αL Integrin chain); CD11b (αM Integrin chain, CR3, Mo1, C3niR, Mac-1); CD11c (αX Integrin, P150, 95, AXb2); CDw17 (Lactosylceramide, LacCer); CD18 (Integrin β2, CD11a, b, c β-subunit); CD19 (B4); CD20 (B1, Bp35); CD21 (CR2, EBV-R, C3dR); CD22 (BL-CAM, Lyb8, Siglec-2); CD23 (FceRII, B6, BLAST-2, Leu-20); CD24 (BBA-1, HSA); CD25 (Tac antigen, IL-2Rα, p55); CD26 (DPP IV ectoeneyme, ADA binding protein); CD27 (T14, S152); CD29 (Platelet GPIIa, β-1 integrin, GP); CD31 (PECAM-1, Endocam); CD32 (FCγRII); CD35 (CR1, C3b/C4b receptor); CD37 (gp52-40); CD38 (ADPribosyl cyclase, T10); CD39 (ATPdehydrogenase, NTPdehydrogenase-1); CD40 (Bp50); CD44 (ECMRII, H-CAM, Pgp-1); CD45 (LCA, T200, B220, Ly5); CD45RA; CD45RB; CD45RC; CD45RO (UCHL-1); CD46 (MCP); CD47 (gp42, IAP, OA3, Neurophilin); CD47R (MEM-133); CD48 (Blast-1, Hulym3, BCM-1, OX-45); CD49b (VLA-2α, gpla, α2 Integrin); CD49c (VLA-3α, α3 Integrin); CD49d (VLA-4α, α4 Integrin); CD50 (ICAM-3); CD52 (CAMPATH-1, HES); CD53 (OX-44); CD54 (ICAM-1); CD55 (DAF); CD58 (LFA-3); CD60a (GD3); CD62L (L-selectin, LAM-1, LECAM-1, MEL-14, Leu8, TQ1); CD72 (Ly-19.2, Ly-32.2, Lyb-2); CD73 (Ecto-5′-nuciotidase); CD74 (Ii, invariant chain); CD75 (sialo-masked Lactosamine); CD75S (α2, 6 sialytated Lactosamine); CD77 (Pk antigen, BLA, CTH/Gb3); CD79a (Igα, MB1); CD79b (Igβ, B29); CD80; CD81 (TAPA-1); CD82 (4F9, C33, IA4, KAI1, R2); CD83 (HB15); CD84 (P75, GR6); CD85j (ILT2, LIR1, MIR7); CDw92 (p70); CD95 (APO-1, FAS, TNFRSF6); CD98 (4F2, FRP-1, RL-388); CD99 (MIC2, E2); CD100 (SEMA4D); CD102 (ICAM-2); CD108 (SEMA7A, JMH blood group antigen); CDw119 (IFNγR, IFNγRa); CD120a (TNFRI, p55); CD120b (TNFRII, p75, TNFR p80); CD121b (Type 2 IL-1R); CD122 (IL2Rβ); CD124 (IL-4Rα); CD130 (gp130); CD132 (Common γ chain, IL-2Rγ); CDw137 (4-1BB, ILA); CD139; CD147 (Basigin, EMMPRIN, M6, OX47); CD150 (SLAM, IPO-3); CD162 (PSGL-1); CD164 (MGC-24, MUC-24); CD166 (ALCAM, KG-CAM, SC-1, BEN, DM-GRASP); CD167a (DDR1, trkE, cak); CD171 (L1CMA, NILE); CD175s (Sialyl-Tn (S-Tn)); CD180 (RP105, Bgp95, Ly64); CD184 (CXCR4, NPY3R); CD185 (CXCR5); CD192 (CCR2); CD196 (CCR6); CD197 (CCR7 (was CDw197)); CDw197 (CCR7, EBI1, BLR2); CD200 (OX2); CD205 (DEC-205); CDw210 (CK); CD213a (CK); CDw217 (CK); CDw218a (IL18Rα); CDw218b (IL18Rβ); CD220 (Insulin R); CD221 (IGF1 R); CD222 (M6P-R, IGFII-R); CD224 (GGT); CD225 (Leu13); CD226 (DNAM-1, PTA1); CD227 (MUC1, PUM, PEM, EMA); CD229 (Ly9); CD230 (Prion Protein (Prp)); CD232 (VESP-R); CD245 (p220/240); CD247 (CD3 Zeta Chain); CD261 (TRAIL-R1, TNF-R superfamily, member 10a); CD262 (TRAIL-R2, TNF-R superfamily, member 10b); CD263 (TRAIL-R3, TNF-R superfamily, member 10c); CD264 (TRAIL-R4, TNF-R superfamily, member 10d); CD265 (TRANCE-ft TNF-R superfamily, member 11a); CD267 (TACI, TNF-R superfamily, member 13B); CD268 (BAFFR, TNF-R superfamily, member 13C); CD269 (BCMA, TNF-R superfamily, member 16); CD275 (B7H2, ICOSL); CD277 (BT3.1.B7 family: Butyrophilin 3); CD295 (LEPR); CD298 (ATP1B3 Na K ATPase β3 subunit); CD300a (CMRF-35H); CD300c (CMRF-35A); CD305 (LAIR1); CD307 (IRTA2); CD315 (CD9P1); CD316 (EW12); CD317 (BST2); CD319 (CRACC, SLAMF7); CD321 (JAM1); CD322 (JAM2); CDw327 (Siglec6, CD33L); CD68 (gp 100, Macrosialin); CXCR5; VLA-4; class II MHC; surface IgM; surface IgD; APRL; and/or BAFF—R; wherein the names listed in parentheses represent alternative names. Examples of markers include those provided elsewhere herein.

In some embodiments, B cell targeting can be accomplished by any targeting moiety that specifically binds to any entity (e.g., protein, lipid, carbohydrate, small molecule, etc.) that is prominently expressed and/or present on B cells upon activation (i.e., activated B cell marker). Exemplary activated B cell markers include, but are not limited to, CD1a (R4, T6, HTA-1); CD1b (R1); CD15s (Sialyl Lewis X); CD15u (3′ sulpho Lewis X); CD15su (6 sulpho-sialyl Lewis X); CD30 (Ber-H2, Ki-1); CD69 (AIM, EA 1, MLR3, gp34/28, VEA); CD70 (Ki-24, CD27 ligand); CD80 (B7, B7-1, BB1); CD86 (B7-2/B70); CD97 (BLKDD/F12); CD125 (IL-5Rα); CD126 (IL-6Rα); CD138 (Syndecan-1, Heparan sulfate proteoglycan); CD152 (CTLA-4); CD252 (OX40L, TNF (ligand) superfamily, member 4); CD253 (TRAIL, TNF (ligand) superfamily, member 10); CD279 (PD1); CD289 (TLR9, TOLL-like receptor 9); and CD312 (EMR2); wherein the names listed in parentheses represent alternative names. Examples of markers include those provided elsewhere herein.

“Chronic infectious agent antigen” means an antigen of an infectious agent that produces a chronic infection that is characterized by a Th2-type pattern of cytokine response or a suboptimal and/or ineffective Th1-type response to the antigen. In an embodiment, immunofeature surfaces according to the invention do not comprise a chronic infectious agent antigen. In embodiments, chronic infectious agent antigens comprise antigens derived from Leishmania parasites, candida albicans, Aspergillus fumigatus, plasmodium parasites, toxoplasma gondii, mycobacteria, HIV, HBV, HCV, EBV, CMV and schistosoma trematodes.

“Co-administer” or “co-administration” means administering inventive synthetic nanocarriers to a subject within 24 or fewer, preferably 12 or fewer, more preferably 6 or fewer hours of administration to that subject of an antigen that is relevant to treatment of the condition. Co-administration may take place through administration in the same dosage form or in separate dosage forms.

“Coupled” means attached to or contained within the synthetic nanocarrier. In some embodiments, the coupling is covalent. In some embodiments, the covalent coupling is mediated by one or more linkers. In some embodiments, the coupling is non-covalent. In some embodiments, the non-covalent coupling is mediated by charge interactions, affinity interactions, metal coordination, physical adsorption, hostguest interactions, hydrophobic interactions, TT stacking interactions, hydrogen bonding interactions, van der Waals interactions, magnetic interactions, electrostatic interactions, dipole-dipole interactions, and/or combinations thereof. In embodiments, the coupling may arise in the context of encapsulation within the synthetic nanocarriers, using conventional techniques. In embodiments, immunostimulatory agents, T cell antigens, and the moieties of which the immunofeature surfaces according to the invention, may each individually or in any combination thereof, be coupled to a synthetic nanocarrier

“Dosage form” means a drug in a medium, carrier, vehicle, or device suitable for administration to a subject.

“Identifying a subject suffering from a condition” means diagnosing or detecting or ascertaining whether a subject has or is likely to have a particular medical condition.

“Immunofeature surface” means a surface that comprises multiple moieties, wherein: (1) the immunofeature surface excludes moieties that are the Fc portion of an antibody; and (2) the moieties are present in an amount effective to provide avidity-based binding to mammalian antigen presenting cells.

Avidity-based binding is binding that is based on an avidity effect (this type of binding may also be referred to as “high avidity” binding). In a preferred embodiment, the presence of an immunofeature surface can be determined using an in vivo assay followed by an in vitro assay as follows (although other methods that ascertain the presence of binding based on an avidity effect (i.e. “high avidity” binding) may be used in the practice of the present invention as well.)

The in vivo assay makes use of two sets of synthetic nanocarriers carrying different fluorescent labels, with one set of synthetic nanocarriers having the immunofeature surface and the other set serving as a control. To test whether the immunofeature surface can target synthetic nanocarriers to Antigen Presenting Cells in vivo, both sets of synthetic nanocarriers are mixed 1:1 and injected into the footpad of a mouse. Synthetic nanocarrier accumulation on dendritic cells and subcapsular sinus macrophages is measured by harvesting the draining popliteal lymph node of the injected mouse at a time point between 1 to 4 hours and 24 hours after nanocarrier injection, respectively. Lymph nodes are processed for confocal fluorescence immunohistology of frozen sections, counterstained with fluorescent antibodies to mouse-CD11c (clone HL3, BD BIOSCIENCES® or mouse-CD169 (clone 3D6.112 from SEROTEC®) and analyzed by planimetry using a suitable image processing software, such as ADOBE® PHOTOSHOP®). Targeting of antigen presenting cells by the immunofeature surface is established if synthetic nanocarriers comprising the immunofeature surface associate with dendritic cells and/or subcapsular sinus macrophages at least 1.2-fold, preferably at least 1.5-fold, more preferably at least 2-fold more frequently than control nanocarriers.

In a preferred embodiment, the in vitro assay that accompanies the in vivo assay determines the immobilization of human or murine dendritic cells or murine subcapsular sinus macrophages (collectively “In Vitro Antigen Presenting Cells”) on a biocompatible surface that is coated with either the moieties of which the immunofeature surface is comprised, or an antibody that is specific for an In Vitro Antigen Presenting Cell-expressed surface antigen (for human dendritic cells: anti-CD1c (BDCA-1) clone AD5-8E7 from Miltenyi BIOTEC®, for mouse dendritic cells: anti-CD11c (αX integrin) clone HL3, BD BIOSCIENCES®, or for murine subcapsular sinus macrophages: anti-CD169 clone 3D6.112 from SEROTEC®) such that (i) an optimal coating density corresponding to maximal immobilization of the In Vitro Antigen Presenting Cells to the surface which has been coated with the moieties of which the immunofeature surface is comprised is either undetectable or at least 10%, preferably at least 20%, more preferably at least 25%, of that observed with the antibody coated surface; and (ii) if immobilization of In Vitro Antigen Presenting Cells by the immunofeature surface is detectable, the immunofeature surface that is being tested supports half maximal binding at a coating density of moieties of which the immunofeature surface is comprised that is at least 2-fold, preferably at least 3-fold, more preferably at least 4-fold higher than the antibody coating density that supports half maximal binding.

Immunofeature surfaces may be positively charged, negatively charged or neutrally charged at pH=7.2-7.4. Immunofeature surfaces may be made up of the same moiety or a mixture of different moieties. In embodiments, the immunofeature surfaces may comprise B cell antigens. Examples of moieties potentially useful in immunofeature surfaces comprise: nicotine and derivatives thereof, methoxy groups, positively charged amine groups (e.g. tertiary amines), sialyllactose, avidin and/or avidin derivatives such as NeutrAvidin, and residues of any of the above. In an embodiment, the moieties of which the immunofeature surface is comprised are coupled to a surface of the inventive nanocarriers. In another embodiment, the immunofeature surface is coupled to a surface of the inventive nanocarriers.

It should be noted that moieties of which immunofeature surfaces are comprised confer high avidity binding. Not all moieties that could be present on a nanocarrier will confer high avidity binding, as defined specifically in this definition, and described generally throughout the present specification. Accordingly, even though a surface may comprise multiple moieties (sometimes referred to as an “array”), this does not mean that such a surface inherently is an immunofeature surface absent data showing that such a surface confers binding according to the present definition and disclosure.

“Immunostimulatory agent” mean an agent that modulates an immune response to an antigen but is not the antigen or derived from the antigen. “Modulate”, as used herein, refers to inducing, enhancing, suppressing, directing, or redirecting an immune response. Such agents include immunostimulatory agents that stimulate (or boost) an immune response to an antigen but is not an antigen or derived from an antigen. Immunostimulatory agents, therefore, include adjuvants. In some embodiments, the immunostimulatory agent is on the surface of the nanocarrier and/or is incorporated within the synthetic nanocarrier. In embodiments, the immunostimulatory agent is coupled to the synthetic nanocarrier.

In some embodiments, all of the immunostimulatory agents of a synthetic nanocarrier are identical to one another. In some embodiments, a synthetic nanocarrier comprises a number of different types of immunostimulatory agents. In some embodiments, a synthetic nanocarrier comprises multiple individual immunostimulatory agents, all of which are identical to one another. In some embodiments, a synthetic nanocarrier comprises exactly one type of immunostimulatory agent. In some embodiments, a synthetic nanocarrier comprises exactly two distinct types of immunostimulatory agents. In some embodiments, a synthetic nanocarrier comprises greater than two distinct types of immunostimulatory agents.

In some embodiments, a synthetic nanocarrier comprises a lipid membrane (e.g., lipid bilayer, lipid monolayer, etc.), wherein at least one type of immunostimulatory agent is coupled with the lipid membrane. In some embodiments, at least one type of immunostimulatory agent is embedded within the lipid membrane. In some embodiments, at least one type of immunostimulatory agent is embedded within the lumen of a lipid bilayer. In some embodiments, a synthetic nanocarrier comprises at least one type of immunostimulatory agent that is coupled with the interior surface of the lipid membrane. In some embodiments, at least one type of immunostimulatory agent is encapsulated within the lipid membrane of a synthetic nanocarrier. In some embodiments, at least one type of immunostimulatory agent may be located at multiple locations of a synthetic nanocarrier. One of ordinary skill in the art will recognize that the preceding examples are only representative of the many different ways in which multiple immunostimulatory agents may be coupled with different locales of synthetic nanocarriers. Multiple immunostimulatory agents may be located at any combination of locales of synthetic nanocarriers.

“Maximum dimension of a synthetic nanocarrier” means the largest dimension of a nanocarrier measured along any axis of the synthetic nanocarrier. “Minimum dimension of a synthetic nanocarrier” means the smallest dimension of a synthetic nanocarrier measured along any axis of the synthetic nanocarrier. For example, for a spheriodal synthetic nanocarrier, the maximum and minimum dimension of a synthetic nanocarrier would be substantially identical, and would be the size of its diameter. Similarly, for a cubic synthetic nanocarrier, the minimum dimension of a synthetic nanocarrier would be the smallest of its height, width or length, while the maximum dimension of a synthetic nanocarrier would be the largest of its height, width or length. In an embodiment, a minimum dimension of at least 75%, preferably at least 80%, more preferably at least 90%, of the synthetic nanocarriers in a sample, based on the total number of synthetic nanocarriers in the sample, is greater than 100 nm. In a embodiment, a maximum dimension of at least 75%, preferably at least 80%, more preferably at least 90%, of the synthetic nanocarriers in a sample, based on the total number of synthetic nanocarriers in the sample, is equal to or less than 5 μm. Preferably, a minimum dimension of at least 75%, preferably at least 80%, more preferably at least 90%, of the synthetic nanocarriers in a sample, based on the total number of synthetic nanocarriers in the sample, is greater than 110 nm, more preferably greater than 120 nm, more preferably greater than 130 nm, and more preferably still greater than 150 nm. Preferably, a maximum dimension of at least 75%, preferably at least 80%, more preferably at least 90%, of the synthetic nanocarriers in a sample, based on the total number of synthetic nanocarriers in the sample is equal to or less than 3 μm, more preferably equal to or less than 2 μm, more preferably equal to or less than 1 μm, more preferably equal to or less than 800 nm, more preferably equal to or less than 600 nm, and more preferably still equal to or less than 500 nm. In preferred embodiments, a maximum dimension of at least 75%, preferably at least 80%, more preferably at least 90%, of the synthetic nanocarriers in a sample, based on the total number of synthetic nanocarriers in the sample, is equal to or greater than 100 nm, more preferably equal to or greater than 120 nm, more preferably equal to or greater than 130 nm, more preferably equal to or greater than 140 nm, and more preferably still equal to or greater than 150 nm. Measurement of synthetic nanocarrier sizes is obtained by suspending the synthetic nanocarriers in a liquid (usually aqueous) media and using dynamic light scattering (e.g. using a Brookhaven ZetaPALS instrument).

“Non-antigenic immunofeature surface” means an immunofeature surface that does not include moieties that activate T cells or B cells when present on the surface of a synthetic nanocarrier, or includes moieties that activate T cells or B cells when present on a surface of a synthetic nanocarrier but in an amount insufficient for the synthetic nanocarrier to activate T cells or B cells. In an embodiment, activation of human and mouse lymphocytes may be detected by analysis of cell surface ‘activation markers’. For instance, CD69 (Very Early Activation Antigen) is a cell surface molecule that is expressed highly on activated T-cells and B-cells but not on resting non-activated cells. Activation of T-cells and B-cells from human peripheral blood mononuclear cells (PBMC) or from mouse spleen may be detected using fluorochrome-conjugated anti-CD69 antibodies and analysis using flow cytometry. Activated lymphocytes show a greater than 2-fold increase in fluorescence intensity over non-activated control lymphocytes. In an embodiment, immunofeature surfaces according to the invention comprise a non-antigenic immunofeature surface.

“Passive administration” means administration of a substance, such as an antigen, by directing, or arranging for, a subject to conduct themselves in a manner that would lead the subject to be exposed to the antigen. For instance, in an embodiment passive administration of an allergen occurs by directing a subject to allow himself or herself to be exposed allergens that are present in the environment (i.e. “environmental allergens”).

“Pharmaceutically acceptable excipient” means a pharmacologically inactive substance added to an inventive composition to further facilitate administration of the composition. Examples, without limitation, of pharmaceutically acceptable excipients include calcium carbonate, calcium phosphate, various diluents, various sugars and types of starch, cellulose derivatives, gelatin, vegetable oils and polyethylene glycols.

“Subject” means an animal, including mammals such as humans and primates; avians; domestic household or farm animals such as cats, dogs, sheep, goats, cattle, horses and pigs; laboratory animals such as mice, rats and guinea pigs; fish; and the like.

“Synthetic nanocarrier(s)” means a discrete object that is not found in nature, and that possesses at least dimension that is less than or equal to 5 microns in size. Albumin nanoparticles are expressly included as synthetic nanocarriers.

A synthetic nanocarrier can be, but is not limited to, one or a plurality of lipid-based nanoparticles, polymeric nanoparticles, metallic nanoparticles, surfactant-based emulsions, dendrimers, buckyballs, nanowires, virus-like particles, peptide or protein-based particles (such as albumin nanoparticles) and/or nanoparticles that are developed using a combination of nanomaterials such as lipid-polymer nanoparticles. Synthetic nanocarriers may be a variety of different shapes, including but not limited to spheroidal, cubic, pyramidal, oblong, cylindrical, toroidal, and the like. Synthetic nanocarriers according to the invention comprise one or more surfaces. Exemplary synthetic nanocarriers that can be adapted for use in the practice of the present invention comprise: (1) the biodegradable nanoparticles disclosed in U.S. Pat. No. 5,543,158 to Gref et al., (2) the polymeric nanoparticles of Published US Patent Application 20060002852 to Saltzman et al., or (4) the lithographically constructed nanoparticles of Published US Patent Application 20090028910 to DeSimone et al. Synthetic nanocarriers according to the invention that have a minimum dimension of equal to or less than about 100 nm, preferably equal to or less than 100 nm, do not comprise a surface with hydroxyl groups that activate complement or alternatively comprise a surface that consists essentially of moieties that are not hydroxyl groups that activate complement. In a preferred embodiment, synthetic nanocarriers according to the invention that have a minimum dimension of equal to or less than about 100 nm, preferably equal to or less than 100 nm, do not comprise a surface that substantially activates complement or alternatively comprise a surface that consists essentially of moieties that do not substantially activate complement. In a more preferred embodiment, synthetic nanocarriers according to the invention that have a minimum dimension of equal to or less than about 100 nm, preferably equal to or less than 100 nm, do not comprise a surface that activates complement or alternatively comprise a surface that consists essentially of moieties that do not activate complement.

“T cell antigen” means any antigen that is recognized by and triggers an immune response in a T cell (e.g., an antigen that is specifically recognized by a T cell receptor on a T cell or an NKT cell via presentation of the antigen or portion thereof bound to a Class I or Class II major histocompatability complex molecule (MHC), or bound to a CD1 complex. In some embodiments, an antigen that is a T cell antigen is also a B cell antigen. In other embodiments, the T cell antigen is not also a B cell antigen. T cells antigens generally are proteins or peptides. T cell antigens may be an antigen that stimulates a CD8+ T cell response, a CD4+ T cell response, or both. The nanocarriers, therefore, in some embodiments can effectively stimulate both types of responses. In some embodiments the T cell antigen is a ‘universal’ T cell antigen (i.e., one which can generate an enhanced response to an unrelated B cell antigen through stimulation of T cell help). In embodiments, a universal T cell antigen may comprise one or more peptides derived from tetanus toxoid, Epstein-Barr virus, influenza virus, or a Padre peptide.

“Th1 biasing immunostimulatory agent” means an immunostimulatory agent that (1) biases an immune response from a response that is characterized by a Th2-type cytokine response to a response that is characterized by a Th1-type cytokine response, or (2) amplifies a suboptimal and/or ineffective Th1-type response.

In certain embodiments, Th1 biasing immunostimulatory agents may be interleukins, interferon, cytokines, etc. In specific embodiments, a Th1 biasing immunostimulatory agent may be a natural or synthetic agonist for a Toll-like receptor (TLR) such as TLR-1, TLR-2, TLR-3, TLR-4, TLR-5, TLR-6, TLR-7, TLR-8, TLR-9, TLR-10, and TLR-11 agonists.

In specific embodiments, synthetic nanocarriers incorporate agonists for toll-like receptors (TLRs) 7 & 8 (“TLR 7/8 agonists”). Of utility are the TLR 7/8 agonist compounds disclosed in U.S. Pat. No. 6,696,076 to Tomai et al., including but not limited to imidazoquinoline amines, imidazopyridine amines, 6,7-fused cycloalkylimidazopyridine amines, and 1,2-bridged imidazoquinoline amines. Preferred Th1 biasing immunostimulatory agents comprise imiquimod and R848.

In specific embodiments, synthetic nanocarriers incorporate a ligand for Toll-like receptor (TLR)-9, such as immunostimulatory DNA molecules comprising CpGs, which induce type I interferon secretion, and stimulate T and B cell activation leading to increased antibody production and cytotoxic T cell responses (Krieg et al., CpG motifs in bacterial DNA trigger direct B cell activation. Nature. 1995. 374:546-549; Chu et al. CpG oligodeoxynucleotides act as adjuvants that switch on T helper 1 (Th1) immunity. J. Exp. Med. 1997. 186:1623-1631; Lipford et al. CpG-containing synthetic oligonucleotides promote B and cytotoxic T cell responses to protein antigen: a new class of vaccine adjuvants. Eur. J. Immunol. 1997. 27:2340-2344; Roman et al. Immunostimulatory DNA sequences function as T helper-1-promoting adjuvants. Nat. Med. 1997. 3:849-854; Davis et al. CpG DNA is a potent enhancer of specific immunity in mice immunized with recombinant hepatitis B surface antigen. J. Immunol. 1998. 160:870-876; Lipford et al., Bacterial DNA as immune cell activator. Trends Microbiol. 1998. 6:496-500. In embodiments, CpGs may comprise modifications intended to enhance stability, such as phosphorothioate linkages, or other modifications, such as modified bases. See, for example, U.S. Pat. Nos. 5,663,153, 6,194,388, 7,262,286, or 7,276,489. In certain embodiments, to stimulate immunity rather than tolerance, a synthetic nanocarrier incorporates an immunostimulatory agent that promotes DC maturation (needed for priming of naive T cells) and the production of cytokines, such as type I interferons, which promote antibody responses and anti-viral immunity. In some embodiments, an immunostimulatory agent may be a TLR-4 agonist, such as bacterial lipopolysacharide (LPS), VSV-G, and/or HMGB-1. In some embodiments, immunostimulatory agents are cytokines, which are small proteins or biological factors (in the range of 5 kD-20 kD) that are released by cells and have specific effects on cell-cell interaction, communication and behavior of other cells. In some embodiments, immunostimulatory agents may be proinflammatory stimuli released from necrotic cells (e.g., urate crystals). In some embodiments, immunostimulatory agents may be activated components of the complement cascade (e.g., CD21, CD35, etc.). In some embodiments, immunostimulatory agents may be activated components of immune complexes. The immunostimulatory agents also include complement receptor agonists, such as a molecule that binds to CD21 or CD35. In some embodiments, the complement receptor agonist induces endogenous complement opsonization of the nanocarrier. Immunostimulatory agents also include cytokine receptor agonists, such as a cytokine.

In some embodiments, the cytokine receptor agonist is a small molecule, antibody, fusion protein, or aptamer. In embodiments, immunostimulatory agents also may comprise immunostimulatory RNA molecules, such as but not limited to dsRNA or poly I:C (a TLR3 stimulant), and/or those disclosed in F. Heil et al., “Species-Specific Recognition of Single-Stranded RNA via Toll-like Receptor 7 and 8” Science 303(5663), 1526-1529 (2004); J. Vollmer et al., “Immune modulation by chemically modified ribonucleosides and oligoribonucleotides” WO 2008033432 A2; A. Forsbach et al., “Immunostimulatory oligoribonucleotides containing specific sequence motif(s) and targeting the Toll-like receptor 8 pathway” WO 2007062107 A2; E. Uhlmann et al., “Modified oligoribonucleotide analogs with enhanced immunostimulatory activity” U.S. Pat. Appl. Publ. US 2006241076; G. Lipford et al., “Immunostimulatory viral RNA oligonucleotides and use for treating cancer and infections” WO 2005097993 A2; G. Lipford et al., “Immunostimulatory G,U-containing oligoribonucleotides, compositions, and screening methods” WO 2003086280 A2.

In some embodiments, the present invention provides pharmaceutical compositions comprising vaccine nanocarriers formulated with one or more adjuvants. The term “adjuvant”, as used herein, refers to an agent that does not constitute a specific antigen, but boosts the immune response to the administered antigen.

In some embodiments, vaccine nanocarriers are formulated with one or more adjuvants such as gel-type adjuvants (e.g., aluminum hydroxide, aluminum phosphate, calcium phosphate, etc.), microbial adjuvants (e.g., immunomodulatory DNA sequences that include CpG motifs; immunostimulatory RNA molecules; endotoxins such as monophosphoryl lipid A; exotoxins such as cholera toxin, E. coli heat labile toxin, and pertussis toxin; muramyl dipeptide, etc.); oil-emulsion and emulsifier-based adjuvants (e.g., Freund's Adjuvant, MF59 [Novartis], SAF, etc.); particulate adjuvants (e.g., liposomes, biodegradable microspheres, saponins, etc.); synthetic adjuvants (e.g., nonionic block copolymers, muramyl peptide analogues, polyphosphazene, synthetic polynucleotides, etc.), and/or combinations thereof.

“Time different from administration” or “a time different from a time when the composition is administered” means a time more than about 30 seconds either before or after administration, preferably more than about 1 minute either before or after administration, more preferably more than 5 minutes either before or after administration, still more preferably more than 1 day either before or after administration, still more preferably more than 2 days either before or after administration, still more preferably more than 1 week either before or after administration, and still more preferably more than 1 month either before or after administration.

“Tumor antigen” means a cell-surface antigen of a tumor that elicits a specific immune response in a subject in which the tumor is present. In an embodiment, immunofeature surfaces according to the invention do not comprise a tumor antigen.

“Vector effect” means the establishment of an unwanted immune response to a synthetic nanocarrier, rather than to an antigen on the synthetic nanocarrier that is relevant to treatment of the condition. Vector effects can occur when the material of the synthetic nanocarrier is capable of stimulating a strong humoral immune response because of its chemical composition or structure. In one circumstance, synthetic carriers that induce a vector effect will ‘flood’ the immune system with antigen other than the antigen that is relevant to treatment of the condition, the result being a weak response to the relevant antigen. In another circumstance the unwanted immune response is a strong response to the nanocarrier itself, such that the nanocarrier is ineffective and, perhaps, even dangerous, on subsequent use in the same subject. In certain embodiments, therefore, the surface(s) of synthetic nanocarriers are not formed principally or substantially from material that provokes a vector effect, such as, for example, virus coat proteins. It should be understood, however, that strongly immunogenic materials such as virus coat proteins can be used to manufacture synthetic nanocarriers of the invention, and, in circumstances where the vector effect is to be avoided, then the synthetic nanocarriers themselves can be modified to reduce or eliminate a vector effect. For example, vector-effect inducing materials (e.g. virus coat proteins used in virus-like particles) may be placed remotely from the surface of the synthetic nanocarrier or coated with immune-altering molecules, such as polyethylene glycols, to render the actual surface of the nanocarrier less immunogenic and thereby avoid vector effects that would otherwise occur.

### C. INVENTIVE IMMUNONANOTHERAPEUTIC COMPOSITIONS

A wide variety of synthetic nanocarriers can be used according to the invention. In some embodiments, synthetic nanocarriers are spheres or spheroids. In some embodiments, synthetic nanocarriers are flat or plate-shaped. In some embodiments, synthetic nanocarriers are cubes or cubic. In some embodiments, synthetic nanocarriers are ovals or ellipses. In some embodiments, synthetic nanocarriers are cylinders, cones, or pyramids.

It is often desirable to use a population of synthetic nanocarriers that is relatively uniform in terms of size, shape, and/or composition so that each synthetic nanocarrier has similar properties. For example, at least 80%, at least 90%, or at least 95% of the synthetic nanocarriers may have a minimum dimension or maximum dimension that falls within 5%, 10%, or 20% of the average diameter or average dimension. In some embodiments, a population of synthetic nanocarriers may be heterogeneous with respect to size, shape, and/or composition.

Synthetic nanocarriers can be solid or hollow and can comprise one or more layers. In some embodiments, each layer has a unique composition and unique properties relative to the other layer(s). To give but one example, synthetic nanocarriers may have a core/shell structure, wherein the core is one layer (e.g. a polymeric core) and the shell is a second layer (e.g. a lipid bilayer or monolayer). Synthetic nanocarriers may comprise a plurality of different layers.

In some embodiments, synthetic nanocarriers may optionally comprise one or more lipids. In some embodiments, a synthetic nanocarrier may comprise a liposome. In some embodiments, a synthetic nanocarrier may comprise a lipid bilayer. In some embodiments, a synthetic nanocarrier may comprise a lipid monolayer. In some embodiments, a synthetic nanocarrier may comprise a micelle. In some embodiments, a synthetic nanocarrier may comprise a core comprising a polymeric matrix surrounded by a lipid layer (e.g., lipid bilayer, lipid monolayer, etc.). In some embodiments, a synthetic nanocarrier may comprise a non-polymeric core (e.g., metal particle, quantum dot, ceramic particle, bone particle, viral particle, proteins, nucleic acids, carbohydrates, etc.) surrounded by a lipid layer (e.g., lipid bilayer, lipid monolayer, etc.).

In some embodiments, synthetic nanocarriers can comprise one or more polymeric matrices. In some embodiments, such a polymeric matrix can be surrounded by a coating layer (e.g., liposome, lipid monolayer, micelle, etc.). In some embodiments, various elements of the synthetic nanocarriers can be coupled with the polymeric matrix.

In some embodiments, an immunofeature surface, targeting moiety, and/or immunostimulatory agent can be covalently associated with a polymeric matrix. In some embodiments, covalent association is mediated by a linker. In some embodiments, an immunofeature surface, targeting moiety, and/or immunostimulatory agent can be noncovalently associated with a polymeric matrix. For example, in some embodiments, an immunofeature surface, targeting moiety, and/or immunostimulatory agent can be encapsulated within, surrounded by, and/or dispersed throughout a polymeric matrix. Alternatively or additionally, an immunofeature surface, targeting moiety, and/or immunostimulatory agent can be associated with a polymeric matrix by hydrophobic interactions, charge interactions, van der Waals forces, etc.

A wide variety of polymers and methods for forming polymeric matrices therefrom are known in the art of drug delivery. In general, a polymeric matrix comprises one or more polymers. Polymers may be natural or unnatural (synthetic) polymers. Polymers may be homopolymers or copolymers comprising two or more monomers. In terms of sequence, copolymers may be random, block, or comprise a combination of random and block sequences. Typically, polymers in accordance with the present invention are organic polymers.

Examples of polymers suitable for use in the present invention include, but are not limited to polyethylenes, polycarbonates (e.g. poly(1,3-dioxan-2one)), polyanhydrides (e.g. poly(sebacic anhydride)), polyhydroxyacids (e.g. poly(β-hydroxyalkanoate)), polypropylfumerates, polycaprolactones, polyamides (e.g. polycaprolactam), polyacetals, polyethers, polyesters (e.g., polylactide, polyglycolide), poly(orthoesters), polycyanoacrylates, polyvinyl alcohols, polyurethanes, polyphosphazenes, polyacrylates, polymethacrylates, polyureas, polystyrenes, and polyamines.

In some embodiments, polymers in accordance with the present invention include polymers which have been approved for use in humans by the U.S. Food and Drug Administration (FDA) under 21 C.F.R. §177.2600, including but not limited to polyesters (e.g., polylactic acid, poly(lactic-co-glycolic acid), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates.

In some embodiments, polymers can be hydrophilic. For example, polymers may comprise anionic groups (e.g., phosphate group, sulphate group, carboxylate group); cationic groups (e.g., quaternary amine group); or polar groups (e.g., hydroxyl group, thiol group, amine group). In some embodiments, a synthetic nanocarrier comprising a hydrophilic polymeric matrix generates a hydrophilic environment within the synthetic nanocarrier. In some embodiments, polymers can be hydrophobic. In some embodiments, a synthetic nanocarrier comprising a hydrophobic polymeric matrix generates a hydrophobic environment within the synthetic nanocarrier. Selection of the hydrophilicity or hydrophobicity of the polymer may have an impact on the nature of materials that are incorporated (e.g. coupled) within the synthetic nanocarrier.

In some embodiments, polymers may be modified with one or more moieties and/or functional groups. A variety of moieties or functional groups can be used in accordance with the present invention. In some embodiments, polymers may be modified with polyethylene glycol (PEG), with a carbohydrate, and/or with acyclic polyacetals derived from polysaccharides (Papisov, 2001, ACS Symposium Series, 786:301).

In some embodiments, polymers may be modified with a lipid or fatty acid group. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEG copolymers and copolymers of lactide and glycolide (e.g., PLA-PEG copolymers, PGA-PEG copolymers, PLGA-PEG copolymers, and derivatives thereof. In some embodiments, polyesters include, for example, polyanhydrides, poly(ortho ester), poly(ortho ester)-PEG copolymers, poly(caprolactone), poly(caprolactone)-PEG copolymers, polylysine, polylysine-PEG copolymers, poly(ethyleneimine), poly(ethylene imine)-PEG copolymers, poly(L-lactide-co-L-lysine), poly(serine ester), poly(4-hydroxy-L-proline ester), poly[α-(4-aminobutyl)-L-glycolic acid], and derivatives thereof.

In some embodiments, a polymer may be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present invention is characterized by a lactic acid:glycolic acid ratio of approximately 85:15, approximately 75:25, approximately 60:40, approximately 50:50, approximately 40:60, approximately 25:75, or approximately 15:85.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, polymers can be cationic polymers. In general, cationic polymers are able to condense and/or protect negatively charged strands of nucleic acids (e.g. DNA, RNA, or derivatives thereof). Amine-containing polymers such as poly(lysine) (Zauner et al., 1998, Adv. Drug Del. Rev., 30:97; and Kabanov et al., 1995, Bioconjugate Chem., 6:7), poly(ethylene imine) (PEI; Boussif et al., 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372) are positively-charged at physiological pH, form ion pairs with nucleic acids, and mediate transfection in a variety of cell lines.

In some embodiments, polymers can be degradable polyesters bearing cationic side chains (Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-Llysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633), and poly(4-hydroxy-L-proline ester) (Putnam et al., 1999, Macromolecules, 32:3658; and Lim et al., 1999, J. Am. Chem. Soc., 121:5633).

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing certain suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732.

In some embodiments, polymers can be linear or branched polymers. In some embodiments, polymers can be dendrimers. In some embodiments, polymers can be substantially cross-linked to one another. In some embodiments, polymers can be substantially free of cross-links. In some embodiments, polymers can be used in accordance with the present invention without undergoing a cross-linking step. It is further to be understood that inventive synthetic nanocarriers may comprise block copolymers, graft copolymers, blends, mixtures, and/or adducts of any of the foregoing and other polymers. Those skilled in the art will recognize that the polymers listed herein represent an exemplary, not comprehensive, list of polymers that can be of use in accordance with the present invention.

In some embodiments, synthetic nanocarriers may not comprise a polymeric component. In some embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

In some embodiments, synthetic nanocarriers may optionally comprise one or more amphiphilic entities. In some embodiments, an amphiphilic entity can promote the production of synthetic nanocarriers with increased stability, improved uniformity, or increased viscosity. In some embodiments, amphiphilic entities can be associated with the interior surface of a lipid membrane (e.g., lipid bilayer, lipid monolayer, etc.). Many amphiphilic entities known in the art are suitable for use in making synthetic nanocarriers in accordance with the present invention. Such amphiphilic entities include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid monoglycerides; fatty acid diglycerides; fatty acid amides; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; poly(ethylene glycol)-400-monostearate; phospholipids; synthetic and/or natural detergents having high surfactant properties; deoxycholates; cyclodextrins; chaotropic salts; ion pairing agents; and combinations thereof. An amphiphilic entity component may be a mixture of different amphiphilic entities. Those skilled in the art will recognize that this is an exemplary, not comprehensive, list of substances with surfactant activity. Any amphiphilic entity may be used in the production of synthetic nanocarriers to be used in accordance with the present invention.

In some embodiments, synthetic nanocarriers may optionally comprise one or more carbohydrates. Carbohydrates may be natural or synthetic. A carbohydrate may be a derivatized natural carbohydrate. In certain embodiments, a carbohydrate comprises monosaccharide or disaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In certain embodiments, a carbohydrate is a polysaccharide, including but not limited to pullulan, cellulose, microcrystalline cellulose, hydroxypropyl methylcellulose (HPMC), hydroxycellulose (HC), methylcellulose (MC), dextran, cyclodextran, glycogen, starch, hydroxyethylstarch, carageenan, glycon, amylose, chitosan, N,O-carboxylmethylchitosan, algin and alginic acid, starch, chitin, heparin, konjac, glucommannan, pustulan, heparin, hyaluronic acid, curdlan, and xanthan. In certain embodiments, the carbohydrate is a sugar alcohol, including but not limited to mannitol, sorbitol, xylitol, erythritol, maltitol, and lactitol.

In an embodiment, the inventive synthetic nanocarriers comprise a polymeric matrix, an immunofeature surface that comprises nicotine, and a Th1 biasing immunostimulatory agent that comprises R848, wherein the R848 is coupled to the synthetic nanocarriers by way of being encapsulated within the synthetic nanocarrier. In an embodiment, an inventive composition comprises the synthetic nanocarriers noted above, combined together with a pharmaceutically acceptable excipient in a dosage form suitable for administration to a subject. In the above embodiments, the synthetic nanocarriers are in the shape of spheroids, with the maximum dimension, minimum dimension, and diameter all being 250 nm on average.

In another embodiment, the inventive synthetic nanocarriers comprise a polymeric matrix, targeting moieties that comprise anti-CD11c antibodies coupled to a surface of the synthetic nanocarriers by adsorption, and a Th1 biasing immunostimulatory agent that comprises R848, wherein the R848 is coupled to the synthetic nanocarriers by way of being encapsulated within the synthetic nanocarrier. In an embodiment, an inventive composition comprises the synthetic nanocarriers noted above, combined together with a pharmaceutically acceptable excipient in a dosage form suitable for administration to a subject. In the above embodiments, the synthetic nanocarriers are in the shape of cylinders, with a maximum dimension of 300 nm and a minimum dimension of 150 nm.

Compositions according to the invention comprise inventive synthetic nanocarriers in combination with pharmaceutically acceptable excipients. The compositions may be made using conventional pharmaceutical manufacturing and compounding techniques to arrive at useful dosage forms. In an embodiment, inventive synthetic nanocarriers are suspended in sterile saline solution for injection together with a preservative.

### D. METHODS OF MAKING AND USING THE INVENTIVE IMMUNONANOTHERAPEUTICS

Synthetic nanocarriers may be prepared using a wide variety of methods known in the art. For example, synthetic nanocarriers can be formed by methods as nanoprecipitation, flow focusing fluidic channels, spray drying, single and double emulsion solvent evaporation, solvent extraction, phase separation, milling, microemulsion procedures, microfabrication, nanofabrication, sacrificial layers, simple and complex coacervation, and other methods well known to those of ordinary skill in the art. Alternatively or additionally, aqueous and organic solvent syntheses for monodisperse semiconductor, conductive, magnetic, organic, and other nanomaterials have been described (Pellegrino et al., 2005, Small, 1:48; Murray et al., 2000, Ann. Rev. Mat. Sci., 30:545; and Trindade et al., 2001, Chem. Mat., 13:3843). Additional methods have been described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755, and also U.S. Pat. Nos. 5,578,325 and 6,007,845).

In certain embodiments, synthetic nanocarriers are prepared by a nanoprecipitation process or spray drying. Conditions used in preparing synthetic nanocarriers may be altered to yield particles of a desired size or property (e.g., hydrophobicity, hydrophilicity, external morphology, “stickiness,” shape, etc.). The method of preparing the synthetic nanocarriers and the conditions (e.g., solvent, temperature, concentration, air flow rate, etc.) used may depend on the materials to be coupled to the synthetic nanocarriers and/or the composition of the polymer matrix.

If particles prepared by any of the above methods have a size range outside of the desired range, particles can be sized, for example, using a sieve.

Coupling can be achieved in a variety of different ways, and can be covalent or non-covalent. Such couplings may be arranged to be on a surface or within an inventive synthetic nanocarrier. Elements of the inventive synthetic nanocarriers (such as moieties of which an immunofeature surface is comprised, targeting moieties, polymeric matrices, and the like) may be directly coupled with one another, e.g., by one or more covalent bonds, or may be coupled by means of one or more linkers. Additional methods of functionalizing synthetic nanocarriers may be adapted from Published US Patent Application 2006/0002852 to Saltzman et al., Published US Patent Application 2009/0028910 to DeSimone et al., or Published International Patent Application WO/2008/127532 A1 to Murthy et al.

Any suitable linker can be used in accordance with the present invention. Linkers may be used to form amide linkages, ester linkages, disulfide linkages, etc. Linkers may contain carbon atoms or heteroatoms (e.g., nitrogen, oxygen, sulfur, etc.). In some embodiments, a linker is an aliphatic or heteroaliphatic linker. In some embodiments, the linker is a polyalkyl linker. In certain embodiments, the linker is a polyether linker. In certain embodiments, the linker is a polyethylene linker. In certain specific embodiments, the linker is a polyethylene glycol (PEG) linker.

In some embodiments, the linker is a cleavable linker. To give but a few examples, cleavable linkers include protease cleavable peptide linkers, nuclease sensitive nucleic acid linkers, lipase sensitive lipid linkers, glycosidase sensitive carbohydrate linkers, pH sensitive linkers, hypoxia sensitive linkers, photo-cleavable linkers, heat-labile linkers, enzyme cleavable linkers (e.g. esterase cleavable linker), ultrasound-sensitive linkers, x-ray cleavable linkers, etc. In some embodiments, the linker is not a cleavable linker.

A variety of methods can be used to couple a linker or other element of a synthetic nanocarrier with the synthetic nanocarrier. General strategies include passive adsorption (e.g., via electrostatic interactions), multivalent chelation, high affinity non-covalent binding between members of a specific binding pair, covalent bond formation, etc. (Gao et al., 2005, Curr. Op. Biotechnol., 16:63). In some embodiments, click chemistry can be used to associate a material with a synthetic nanocarrier.

Non-covalent specific binding interactions can be employed. For example, either a particle or a biomolecule can be functionalized with biotin with the other being functionalized with streptavidin. These two moieties specifically bind to each other noncovalently and with a high affinity, thereby associating the particle and the biomolecule. Other specific binding pairs could be similarly used. Alternately, histidine-tagged biomolecules can be associated with particles conjugated to nickel-nitrolotriaceteic acid (Ni-NTA).

For additional general information on coupling, see the journal Bioconjugate Chemistry, published by the American Chemical Society, Columbus Ohio, PO Box 3337, Columbus, Ohio, 43210; “Cross-Linking,” Pierce Chemical Technical Library, available at the Pierce web site and originally published in the 1994-95 Pierce Catalog, and references cited therein; Wong S S, Chemistry of Protein Conjugation and Cross-linking, CRC Press Publishers, Boca Raton, 1991; and Hermanson, G. T., Bioconjugate Techniques, Academic Press, Inc., San Diego, 1996.

Alternatively or additionally, synthetic nanocarriers can be coupled to immunofeature surfaces, targeting moieties, immunostimulatory agents, and/or other elements directly or indirectly via non-covalent interactions. Non-covalent interactions include but are not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. Such couplings may be arranged to be on a surface or within an inventive synthetic nanocarrier.

It is to be understood that the compositions of the invention can be made in any suitable manner, and the invention is in no way limited to compositions that can be produced using the methods described herein. Selection of an appropriate method may require attention to the properties of the particular moieties being associated.

In some embodiments, inventive synthetic nanocarriers are manufactured under sterile conditions. This can ensure that resulting composition are sterile and non-infectious, thus improving safety when compared to non-sterile compositions. This provides a valuable safety measure, especially when subjects receiving synthetic nanocarriers have immune defects, are suffering from infection, and/or are susceptible to infection. In some embodiments, inventive synthetic nanocarriers may be lyophilized and stored in suspension or as lyophilized powder depending on the formulation strategy for extended periods without losing activity.

The inventive compositions may be administered by a variety of routes of administration, including but not limited to parenteral (such as subcutaneous, intramuscular, intravenous, or intradermal); oral; transnasal, transmucosal, rectal; ophthalmic, or transdermal.

Indications treatable using the inventive compositions include but are not limited to those indications in which a biasing from a Th2 pattern of cytokine release towards a Th1 pattern of cytokine release is desirable. Such indications comprise atopic conditions such as but not limited to allergy, allergic asthma, or atopic dermatitis; asthma; chronic obstructive pulmonary disease (COPD, e.g. emphysema or chronic bronchitis); and chronic infections due to chronic infectious agents such as chronic Leishmaniasis, candidiasis or schistosomiasis and infections caused by plasmodia, toxoplasma gondii, mycobacteria, HIV, HBV, HCV EBV or CMV, or any one of the above, or any subset of the above.

Other indications treatable using the inventive compositions include but are not limited to indications in which a subject's Th1 response is suboptimal and/or ineffective. Use of the present invention can enhance a subject's Th1 immune response. Such indications comprise various cancers, and populations with compromised or suboptimal immunity, such as infants, the elderly, cancer patients, individuals receiving immunosuppressive drugs or irradiation, hemodialysis patients and those with genetic or idiopathic immune dysfunction.

It is an aspect of the present invention that the inventive compositions operate in a different way from conventional immunotherapies. In conventional immunotherapies, antigen and immunostimulatory agents are co-administered.

In contrast, in embodiments of the present invention, antigens to which an adaptive immune response is desired are not incorporated into the inventive compositions. In preferred embodiments, such antigens are excluded from the inventive immunofeature surfaces, such that the immunofeature surface do not comprise an antigen that is relevant to treatment of the condition.

Further, in embodiments of the present invention, administration of the inventive compositions do not further comprise administration of an antigen that is relevant to treatment of the condition, either coupled to the nanocarriers or not coupled to the nanocarriers.

In certain embodiments, antigen(s) to which a Th1 biased response is desired are administered at a time different from administration of the composition; wherein administration of the antigen comprises passive administration or active administration.

In each instance, it is unexpected that administration of one or more immunostimulatory agents separated in time from administration of one or more antigens provides a Th1 biased response to administration of the one or more antigens.

### E. EXAMPLES

### Example 1

### PLA-R848 Conjugates

To a two necked round bottom flask equipped with a stir bar and condenser was added the imidazoquinoline resiquimod (R-848, 100 mg, 3.18×10⁻⁴ moles), D/L lactide (5.6 gm, 3.89×10⁻² moles) and anhydrous sodium sulfate (4.0 gm). The flask and contents were dried under vacuum at 50° C. for 8 hours. The flask was then flushed with argon and toluene (100 mL) was added. The reaction was stirred in an oil bath set at 120° C. until all of the lactide had dissolved and then tin ethylhexanoate (75 mg, 60 μL) was added via pipette. Heating was then continued under argon for 16 hours. After cooling, water (20 mL) was added and stirring was continued for 30 minutes. The reaction was diluted with additional toluene (200 mL) and was then washed with water (200 mL). The toluene solution was then washed in turn with 10% sodium chloride solution containing 5% conc. Hydrochloric acid (200 mL) followed by saturated sodium bicarbonate (200 mL). TLC (silica, 10% methanol in methylene chloride) showed that the solution contained no free R-848. The solution was dried over magnesium sulfate, filtered and evaporated under vacuum to give 3.59 grams of polylactic acid-R-848 conjugate. A portion of the polymer was hydrolyzed in base and examined by HPLC for R-848 content. By comparison to a standard curve of R-848 concentration vs HPLC response, it was determined that the polymer contained 4.51 mg of R-848 per gram of polymer. The molecular weight of the polymer was determined by GPC to be about 19,000.

### Example 2

### Nicotine-PEG-PLA Conjugates

A 3-nicotine-PEG-PLA polymer was synthesized as follows:

First, monoamino poly(ethylene glycol) from JenKem® with a molecular weight of 3.5 KD (0.20 gm, 5.7×10-5 moles) and an excess of 4-carboxycotinine (0.126 gm, 5.7×10-4 moles) were dissolved in dimethylformamide (5.0 mL). The solution was stirred and dicyclohexylcarbodiimide (0.124 gm, 6.0×10-4 moles) was added. This solution was stirred overnight at room temperature. Water (0.10 mL) was added and stirring was continued for an additional 15 minutes. The precipitate of dicyclohexyl urea was removed by filtration and the filtrates were evaporated under vacuum. The residue was dissolved in methylene chloride (4.0 mL) and this solution was added to diethyl ether (100 mL). The solution was cooled in the refrigerator for 2 hours and the precipitated polymer was isolated by filtration. After washing with diethyl ether, the solid white polymer was dried under high vacuum. The yield was 0.188 gm. This polymer was used without further purification for the next step.

The cotinine/PEG polymer (0.20 gm, 5.7×10-5 moles) was dissolved in dry tetrahydrofuran (10 mL) under nitrogen and the solution was stirred as a solution of lithium aluminum hydride in tetrahydrofuran (1.43 mL of 2.0M, 2.85×10-3 moles) was added. The addition of the lithium aluminum hydride caused the polymer to precipitate as a gelatinous mass. The reaction was heated to 80° C. under a slow stream of nitrogen and the tetrahydrofuran was allowed to evaporate. The residue was then heated at 80° C. for 2 hours. After cooling, water (0.5 mL) was cautiously added. Once the hydrogen evolution had stopped, 10% methanol in methylene chloride (50 mL) was added and the reaction mixture was stirred until the polymer had dissolved. This mixture was filtered through Celite® brand diatomaceous earth (available from EMD Inc. as Celite® 545, part # CX0574-3) and the filtrates were evaporated to dryness under vacuum. The residue was dissolved in methylene chloride (4.0 mL) and this solution was slowly added to diethyl ether (100 mL). The polymer separated as a white flocculent solid and was isolated by centrifugation. After washing with diethyl ether, the solid was dried under vacuum. The yield was 0.129 gm.

Next, a 100 mL round bottom flask, equipped with a stir bar and reflux condenser was charged with the PEG/nicotine polymer (0.081 gm, 2.2×10-5 moles), D/L lactide (0.410 gm, 2.85×10-3 moles) and anhydrous sodium sulfate (0.380 gm). This was dried under vacuum at 55° C. for 8 hours. The flask was cooled and flushed with argon and then dry toluene (10 mL) was added. The flask was placed in an oil bath set at 120° C., and once the lactide had dissolved, tin ethylhexanoate (5.5 mg, 1.36×10-5 moles) was added. The reaction was allowed to proceed at 120° C. for 16 hours. After cooling to room temperature, water (15 mL) was added and stirring was continued for 30 minutes. Methylene chloride (200 mL) was added, and after agitation in a separatory funnel, the phases were allowed to settle. The methylene chloride layer was isolated and dried over anhydrous magnesium sulfate. After filtration to remove the drying agent, the filtrates were evaporated under vacuum to give the polymer as a colorless foam. The polymer was dissolved in tetrahydrofuran (10 mL) and this solution was slowly added to water (150 mL) with stirring. The precipitated polymer was isolated by centrifugation and the solid was dissolved in methylene chloride (10 mL). The methylene chloride was removed under vacuum and the residue was dried under vacuum. 3-nicotine-PEG-PLA polymer yield was 0.38 gm.

### Example 3

### Prophetic Nanocarrier Formulation—Allergy

Resiquimod (aka R848) is synthesized according to the synthesis provided in Example 99 of U.S. Pat. No. 5,389,640 to Gerster et al. PLA-PEG-nicotine conjugate is prepared according to Example 2. PLA is prepared by a ring opening polymerization using D,L-lactide (MW=approximately 15 KD-18 KD). The PLA structure is confirmed by NMR. The polyvinyl alcohol (Mw=11 KD-31 KD, 85% hydrolyzed) is purchased from VWR scientific. Ovalbumin peptide 323-339 is obtained from Bachem Americas Inc. (3132 Kashiwa Street, Torrance Calif. 90505. Part # 4064565). These were used to prepare the following solutions:

1. Resiquimod in methylene chloride @ 7.5 mg/mL
2. PLA-PEG-nicotine in methylene chloride @ 100 mg/mL
3. PLA in methylene chloride @ 100 mg/mL
4. Ovalbumin peptide 323-339 in water @ 10 mg/mL
5. Polyvinyl alcohol in water @50 mg/mL.

Solution #1 (0.4 mL), solution #2 (0.4 mL), solution #3 (0.4 mL) and solution #4 (0.1 mL) are combined in a small vial and the mixture is sonicated at 50% amplitude for 40 seconds using a Branson Digital Sonifier 250. To this emulsion is added solution #5 (2.0 mL) and sonication at 35% amplitude for 40 seconds using the Branson Digital Sonifier 250 forms the second emulsion. This is added to a beaker containing water (30 mL) and this mixture is stirred at room temperature for 2 hours to form the nanocarriers. A portion of the nanocarrier dispersion (1.0 mL) is diluted with water (14 mL) and this is concentrated by centrifugation in an Amicon Ultra centrifugal filtration device with a membrane cutoff of 100 KD. When the volume is about 250 μL, water (15 mL) is added and the particles are again concentrated to about 250 μL using the Amicon device. A second washing with phosphate buffered saline (pH=7.5, 15 mL) is done in the same manner and the final concentrate is diluted to a total volume of 1.0 mL with phosphate buffered saline. This gives a final nanocarrier dispersion of about 2.7 mg/mL in concentration.

The synthetic nanocarriers are then administered to a subject by intramuscular injection. The subject is directed to allow themselves subsequently to be exposed to environmental allergens, such as ragweed pollen. After exposure to environmental allergen, the subject is challenged by another exposure to environmental allergen. Any generation of a Th1-biased response to the environmental allergen challenge is noted.

### Example 4

### Prophetic Nanocarrier Formulation—Allergy

Resiquimod (aka R848) is synthesized according to the synthesis provided in Example 99 of U.S. Pat. No. 5,389,640 to Gerster et al. Carboxylated polylactic acid is prepared using a ring opening polymerization of D,L-lactide that results in PLA-COON (target MW=15-18 KD). The structure is confirmed by NMR. PLA-PEG-methoxy polymer is prepared using methoxy-PEG (polyethylene glycol methyl ether, Item 20509 from Aldrich Chemical, approximately MW of PEG=2 KD) which is used to initiate a ring opening polymerization of D,L-lactide (final polymer MW target=18-20 KD). The structure is confirmed by NMR. Ovalbumin peptide 323-339 is obtained from Bachem Americas Inc. (3132 Kashiwa Street, Torrance Calif. 90505. Part # 4064565). The polyvinyl alcohol (Mw=11 KD-31 KD, 85% hydrolyzed) is purchased from VWR scientific. These are used to prepare the following solutions:

1. Resiquimod in methylene chloride @ 7.5 mg/mL
2. PLA-PEG-methoxy in methylene chloride @ 100 mg/mL
3. PLA-COON in methylene chloride @ 100 mg/mL
4. Ovalbumin peptide 323-339 in water @ 10 mg/mL
5. Polyvinyl alcohol in water @50 mg/mL.

Solution #1 (0.4 mL), solution #2 (0.4 mL), solution #3 (0.4 mL) and solution #4 (0.1 mL) are combined in a small vial and the mixture is sonicated by a Branson Digital Sonifier 250 at 50% amplitude for 40 seconds. To this emulsion is added solution #5 (2.0 mL) and sonication at 35% amplitude for 40 seconds using the Branson Digital Sonifier 250 forms the second emulsion. This is added to a beaker containing water (30 mL) and this mixture is stirred at room temperature for 2 hours to form the nanocarriers. A portion of the nanocarrier dispersion (1.0 mL) is diluted with water (14 mL) and this is concentrated by centrifugation in an Amicon Ultra centrifugal filtration device with a membrane cutoff of 100 KD. When the volume is about 250 μL, water (15 mL) is added and the particles are again concentrated to about 250 μL, using the Amicon device. A second washing with phosphate buffered saline (pH=6.5, 15 mL) is done in the same manner and the final concentrate is diluted to a total volume of 5.0 mL with phosphate buffered saline (pH=6.5). This gives a final nanocarrier dispersion of about 0.6 mg/mL in concentration. To the nanocarrier dispersion is added N-(3-dimethylaminopropyl)-N′-ethylcarbodiimide hydrochloride (EDC, 200 mg) and N-hydroxysuccinimide (NHS, 70 mg) and this mixture is incubated at room temperature for ½ hour. The nanocarriers are washed three times with PBS by centrifugation. After the last washing, the particles are diluted to a volume of 1.0 mL with PBS to give a suspension of NHS-activated nanocarriers with an approximate concentration of 3.0 mg/mL. To this suspension is added anti-CD11c antibody (50 μL @ 5 μg/mL, anti-CD11c antibody clone MJ4-27G12 available from Miltenyi Biotec). The suspension is incubated in a refrigerator overnight. The resulting substituted nanocarriers are washed three times by centrifugation in PBS. After the last washing, the particles are diluted to a volume of 1.0 mL with PBS to give a suspension of anti-CD169 substituted nanocarriers with an approximate concentration of 2.7 mg/mL.

The synthetic nanocarriers are then administered to a subject by intramuscular injection. The subject is directed to allow themselves subsequently to be exposed to environmental allergens, such as ragweed pollen. After exposure to environmental allergen, the subject is challenged by another exposure to environmental allergen. Any generation of a Th1-biased response to the environmental allergen challenge is noted.

### Example 5

### Prophetic Nanocarrier Formulation—Allergy

Synthetic trapezoidal nanocarriers are prepared according to the modified teachings of US Published Patent Application 2009/0028910 as follows:

A patterned perfluoropolyether (PFPE) mold is generated by pouring PFPE-dimethacrylate (PFPE-DMA) containing 1-hydroxycyclohexyl phenyl ketone over a silicon substrate patterned with 200-nm trapezoidal shapes. A poly(dimethylsiloxane) mold is used to confine the liquid PFPE-DMA to the desired area. The apparatus is then subjected to UV light (365 nm) for 10 minutes while under a nitrogen purge. The fully cured PFPE-DMA mold is then released from the silicon master. Separately, a poly(ethylene glycol) (PEG) diacrylate (n=9) is blended with 1 wt % of a photoinitiator, 1-hydroxycyclohexyl phenyl ketone. Resiquimod (R848, synthesized according to the synthesis provided in Example 99 of U.S. Pat. No. 5,389,640 to Gerster et al.) is added at an amount of 1 wt %, based on total polymer weight in the nanocarrier, is added to this PEG-diacrylate monomer solution and the combination is mixed thoroughly. Flat, uniform, non-wetting surfaces are generated by treating a silicon wafer cleaned with “piranha” solution (1:1 concentrated sulfuric acid:30% hydrogen peroxide (aq) solution) with trichloro(1H,1H,2H,2H-perfluorooctyl)silane via vapor deposition in a desiccator for 20 minutes. Following this, 50 μL of the PEG diacrylate/R848/toxoid solution is then placed on the treated silicon wafer and the patterned PFPE mold placed on top of it. The substrate is then placed in a molding apparatus and a small pressure is applied to push out excess PEG-diacrylate/R848/toxoid solution. The entire apparatus is then subjected to UV light (365 nm) for ten minutes while under a nitrogen purge. The synthetic nanocarriers are then removed from the mold and added to a flask with a solution of 5 wt % carbonyldiimidazole in acetone. The synthetic nanocarriers are gently agitated for 24 hours, following which the synthetic nanocarriers are separated from the acetone solution and suspended in water at room temperature. To this suspension is added an excess of anti-CD11c antibody (clone MJ4-27G12 available from Miltenyi Biotec) and the suspension is heated to 37 Deg C. and agitated gently for 24 hours. The labeled synthetic nanocarriers are then separated from the suspension.

The synthetic nanocarriers are then administered to a subject by intramuscular injection. The subject is directed to allow themselves subsequently to be exposed to environmental allergens, such as ragweed pollen. After exposure to environmental allergen, the subject is challenged by another exposure to environmental allergen. Any generation of a Th1-biased response to the environmental allergen challenge is noted.

### Example 6

### Prophetic Nanocarrier Formulation—Cancer

Resiquimod (aka R848) is synthesized according to the synthesis provided in Example 99 of U.S. Pat. No. 5,389,640 to Gerster et al. PLA is prepared by a ring opening polymerization using D,L-lactide (MW=approximately 15 KD-18 KD). The structure is confirmed by NMR. PLA-PEG-methoxy polymer is prepared using methoxy-PEG (polyethylene glycol methyl ether, Item 20509 from Aldrich Chemical, approximately MW of PEG=2 KD) which is used to initiate a ring opening polymerization of D,L-lactide (final polymer MW target=18-20 KD). The structure is confirmed by NMR. Ovalbumin peptide 323-339 is obtained from Bachem Americas Inc. (3132 Kashiwa Street, Torrance Calif. 90505. Part # 4064565). The polyvinyl alcohol (Mw=11 KD-31 KD, 85% hydrolyzed) is purchased from VWR scientific. These are used to prepare the following solutions:

1. Resiquimod in methylene chloride @ 7.5 mg/mL
2. PLA-PEG-methoxy in methylene chloride @ 100 mg/mL
3. PLA in methylene chloride @ 100 mg/mL
4. Ovalbumin peptide 323-339 in water @ 10 mg/mL
5. Polyvinyl alcohol in water @50 mg/mL.

Solution #1 (0.4 mL), solution #2 (0.4 mL), solution #3 (0.4 mL) and solution #4 (0.1 mL) are combined in a small vial and the mixture is sonicated using a Branson Digital Sonifier 250 at 50% amplitude for 40 seconds. To this emulsion is added solution #5 (2.0 mL) and sonication at 35% amplitude for 40 seconds using the Branson Digital Sonifier 250 forms the second emulsion. This is added to a beaker containing water (30 mL) and this mixture is stirred at room temperature for 2 hours to form the nanocarriers. A portion of the nanocarrier dispersion (1.0 mL) is diluted with water (14 mL) and this is concentrated by centrifugation in an Amicon Ultra centrifugal filtration device with a membrane cutoff of 100 KD. When the volume is about 250 μL, water (15 mL) is added and the particles are again concentrated to about 250 μL using the Amicon device. A second washing with phosphate buffered saline (pH=7.5, 15 mL) is done in the same manner and the final concentrate is diluted to a total volume of 1.0 mL with phosphate buffered saline. This gives a final nanocarrier dispersion of about 2.7 mg/mL in concentration.

The synthetic nanocarriers are then administered by intramuscular injection to a subject having a solid tumor. Forty-eight hours following the injection of the synthetic nanocarriers, the subject is exposed to sufficient radiation to cause disruption of the solid tumor. Generation of any anti-tumor cytotoxic T-cells is noted.

### Example 7

### Prophetic Nanocarrier Formulation—Chronic Leishmaniasis

Synthetic nanocarriers are prepared according to the modified teachings of US Published Patent Application 20060002852 as follows:

Avidin at 10 mg/ml is reacted with 10-fold excess of NHS-Palmitic acid in PBS containing 2% deoxycholate buffer. The mixture is sonicated briefly and gently mixed at 37 Deg. C. for 12 hours. To remove excess fatty acid and hydrolyzed ester, reactants are dialyzed against PBS containing 0.15% deoxycholate.

A modified double emulsion method is used for preparation of fatty acid PLGA particles. In this procedure, Resiquimod (R848, synthesized according to the synthesis provided in Example 99 of U.S. Pat. No. 5,389,640 to Gerster et al.) is added at an amount of 1 wt %, based on total polymer weight in the nanocarrier, in 100 μL of PBS, is added drop wise to a vortexing PLGA solution (100 mg PLGA in 2 ml MeCl₂). This mixture is then sonicated on ice three times in 10-second intervals. At this point, 4 ml of an avidin-palmitate/PVA mixture (2 ml avidin-palmitate in 2 ml of 5% PVA) are slowly added to the PLGA solution. This is then sonicated on ice three times in 10-second intervals. After sonication, the material is added drop-wise to a stirring 100 ml of 0.3% PVA. This undergoes vigorous stirring for 4 hours at constant room temperature to evaporate methylene chloride. The resultant emulsion is then purified by centrifugation at 12,000 g for 15 minutes then washed 3× with DI water.

Biotinylated anti-CD11c antibody is prepared as follows. Biotin-NHS is dissolved in DMSO at 1 mg/ml just before use. Anti-CD11c antibody (clone MJ4-27G12 available from Miltenyi Biotec) is added to the solution at a 1/10 dilution, and is incubated on ice for 30 minutes or room temperature for 2 hours at a pH of 7.5-8.5 for biotin-NHS. PBS or HEPES may be used as buffers. The reaction is quenched with Tris.

The resulting synthetic nanocarriers are then suspended in water at room temperature and an excess of biotinylated anti-CD169 antibody (50 μL @ 5 μg/mL, prepared as set forth above) is added to the suspension. The suspension is heated to 37 Deg C. and agitated gently for 24 hours. The labeled synthetic nanocarriers are then separated from the suspension.

The synthetic nanocarriers are then administered by intramuscular injection to a subject suffering from chronic Leishmaniasis that is characterized by a Th2-biased pattern of cytokine expression. Generation of any appropriate antibodies is noted.

### Example 8

### Treatment of Asthma Using Nanocarriers with R848

Synthetic Nanocarriers containing R848 were used to determine whether R848-containing nanocarriers can be used to modify the asthma response from a Th2 phenotype to a Th1 phenotype. Mice (BALB/c; 5 mice per group) were presensitized to ovalbumin on days 0 and 14 with 20 μg ovalbumin and 2 mg Imject® alum (Pierce, Rockford, Ill.) in 200 μL PBS intraperitoneally (i.p.) (groups 3-9; see Tables 1 and 2 for explanation of experimental groups of mice and respective treatments including nanocarrier composition). Control mice received either 200 μL PBS (group 1) or 2 mg Imject® alum in 200 μL PBS i.p (group 2). On days 27, 28, and 29, mice were treated with either PBS (negative control for treatment) (groups 1-4), CpG (OD 1826, 30 μg in 100 μL i.p.; positive control for treatment) (group 5), nicotine-nanocarriers with R848 (100 μg in 100 μL i.p.) (group 6), nicotine-nanocarriers with R848 (100 μg in 60 μL intranasally (i.n.)) (group 7), nicotine-nanocarriers without R848 (100 μg in 100 μL i.p.) (group 8), or nicotine-nanocarriers without R848 (100 μg in 60 μL i.n.) (group 9). Nicotine-nanocarriers with R848 contained 4.4% R848. R848 was conjugated to PLGA (Mw 4.1 kD). The nanocarrier polymer composition was made generally according to the teachings of Examples 1-3, and included 25% PLA-PEG-nicotine and 75% PLA polymer (either R202H from Boehringer Ingelheim or 100 DL 2A from Lakeshore Biomaterials; both version have Mw of 20 kD and free-carboxylic acid termini).

For measurement of lung leukocyte infiltration, mice were challenged with 50 μg ovalbumin in 60 μL PBS i.n. (groups 2 and 4-9) on days 28, 29, and 30. Control mice (groups 1 and 3) received 60 μL PBS i.n. On day 32, 48 hours after the last ovalbumin challenge, mice were euthanized and samples were collected. For cytokine analysis, samples were collected on day 31, 18 hours after the last ovalbumin challenge. Lungs were lavaged 3 times with 1 mL of PBS containing 3 mM EDTA to collect bronchial alveolar lavage fluid (BALF) for cytospins for differential cell counts and for cytokine analysis. Cytospin slides of BALF were stained with Diff-Quik (Dade Behring) and differential cell counts were done. The remainder of the BALF was stored at −20° C. until needed for cytokine analysis. BALF cytokines (IL-12p40, IL-4, IL-13, and IL-5) were measured by ELISA following the manufacturers' (BD Biosciences and R & D Systems) instructions.

TABLE 1
Treatment groups for induction and/or treatment of
asthma. Group 8 treated with nicotine-nanocarriers
(without R848) i.p. for 48 hour experiment or R848
(50 μg in 100 μL) for 18 hour cytokine experiment.

| Group. # | | Treatment | Challenge |
|---|---|---|---|
| (5 mice/ | Sensitization | Injection | Injection |
| group) | Injection route | route | route |
| 1 | PBS (200 μL); i.p. | PBS | PBS |
| | | i.n. | i.n. |
| 2 | Alum (2 mg) in 200 μL PBS; i.p. | PBS | OVA |
| | | i.n. | i.n. |
| 3 | OVA (20 μg) + Alum (2 mg) in | PBS | PBS |
| | 200 μL PBS; i.p. | i.n. | i.n. |
| 4 | OVA (20 μg) + Alum (2 mg) in | PBS | OVA |
| | 200 μL PBS; i.p. | i.n. | i.n. |
| 5 | OVA (20 μg) + Alum (2 mg) in | CpG (30 μg | OVA |
| | 200 μL PBS; i.p. | in 100 μL) | i.n. |
| | | i.p. | |
| 6 | OVA (20 μg) + Alum (2 mg) in | Nic-NP | OVA |
| | 200 μL PBS; i.p. | w/R848 | i.n. |
| | | i.p. | |
| 7 | OVA (20 μg) + Alum (2 mg) in | Nic-NP | OVA |
| | 200 μL PBS; i.p. | w/R848 | i.n. |
| | | i.n. | |
| 8 | OVA (20 μg) + Alum (2 mg) in | Nic-NP | OVA |
| | 200 μL PBS; i.p. | (no R848) | i.n. |
| | | i.p. (48 hr | |
| | | experiment) | |
| | | OR | |
| | | R848 (50 μg | |
| | | in 100 μL) | |
| | | i.p. (18 hr | |
| | | experiment) | |
| 9 | OVA (20 μg) + Alum (2 mg) in | Nic-NP | OVA |
| | 200 μL PBS; i.p. | (no R848) | i.n. |
| | | i.n. | |

TABLE 2
Composition of nanocarriers used for treatment of asthma.

| Nanocarrier lot number | S0864-66-3 | S0845-3-2 |
|---|---|---|
| (Mouse treatment groups) | (Groups 6 & 7) | (Groups 8 & 9) |
| Peptide | None | None |
| TLR agonist (R848) | S0833-78A | None |
| | R848 (50%) | |
| PLA-PEG-Nic | S0835-33 (25%) | S0835-04 (25%) |
| Bulking Polymer | 100 DL 2A (25%) | R2O2H (75%) |

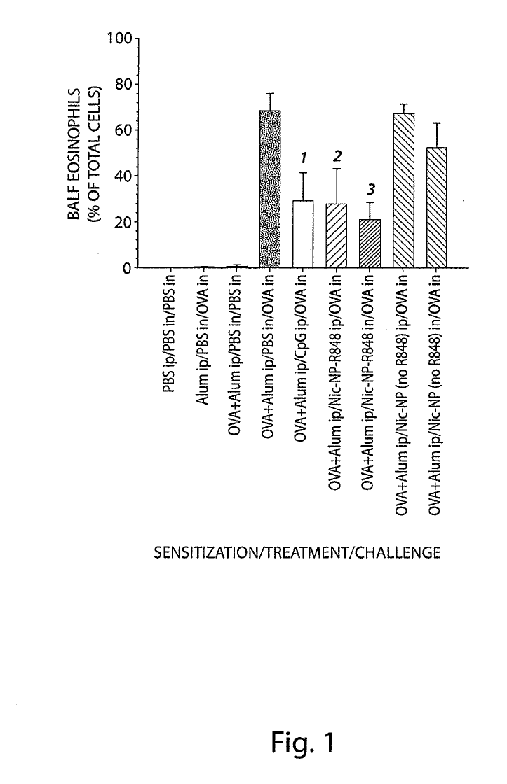

Results: Differential cell counts were done to determine the relative number of eosinophils present in the BALF 48 hours after the last ovalbumin challenge. Mice presensitized to ovalbumin and challenged with ovalbumin (group 4) had a significant influx of eosinophils into the BALF at 48 hours after the final challenge (68.4%±7.6% of total cells) compared to control mice (groups 1, 2, and 3; less than 1% eosinophils of total cells) (p<0.0001; FIG. 1). Treatment with CpG i.p. (group 5) led to a significant reduction in eosinophils (29.2%±12.4%) after challenge with ovalbumin compared to mice presensitized to ovalbumin and challenged with ovalbumin (p<0.0001; FIG. 1). Treatment with nanocarriers with R848 either i.p. (group 6) or i.n. (group 7) led to a significant reduction in eosinophils (28.0%±15.2% and 21.2%±7.3%, respectively) after challenge with ovalbumin compared to mice presensitized to ovalbumin and challenged with ovalbumin (p<0.0001; FIG. 1). Treatment with nanocarriers (without R848) either i.p. (group 8) or i.n. (group 9) did not affect eosinophil influx (67.3%±4.1% and 52.5%±10.7%, respectively) compared to mice presensitized to ovalbumin and challenged with ovalbumin (p>0.05; FIG. 1).

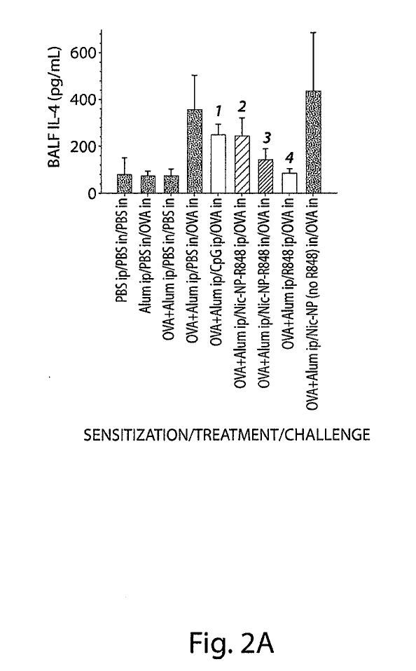

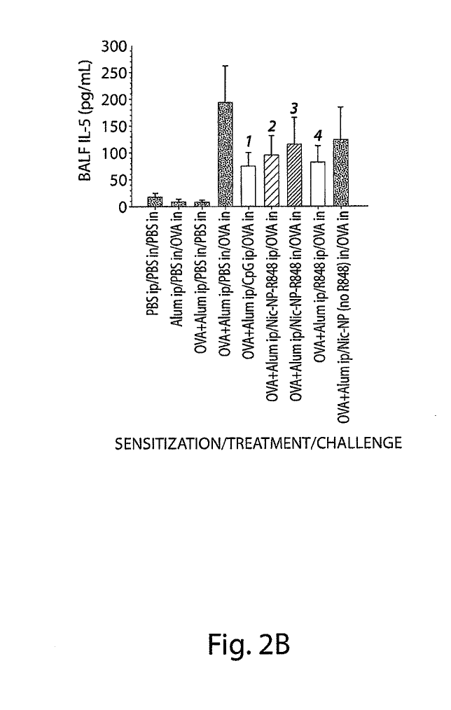

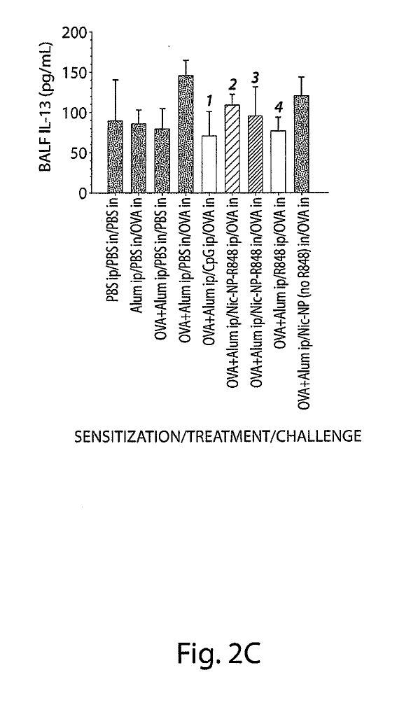

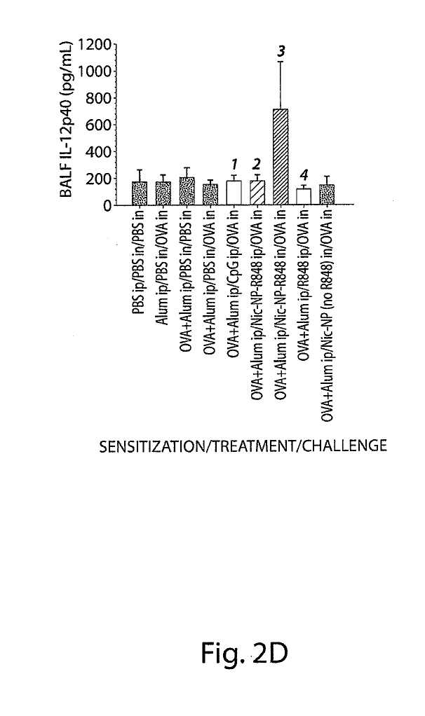

BALF cytokine levels were measured 18 hours after the final ovalbumin challenge. Th2 cytokines (IL-4, IL-5, and IL-13) and Th1 cytokines (IL-12p40) were measured to determine whether treatment led to a shift in cytokine expression from a Th2 cytokine profile to a Th1 cytokine profile. Mice presensitized to ovalbumin and challenged with ovalbumin (group 4) had increased levels of IL-4, IL-5, and IL-13 compared to control mice (groups 1, 2, and 3) (FIG. 2A-C). Treatment with CpG i.p. (group 5) or R848 i.p. (group 8) led to reduced BALF levels of IL-4, IL-5, and IL-13 after challenge with ovalbumin compared to mice presensitized to ovalbumin and challenged with ovalbumin (FIG. 2A-C). Treatment with nanocarriers with R848 either i.p. (group 6) or i.n. (group 7) led to reduced levels of BALF IL-4, IL-5, and IL-13 after challenge with ovalbumin compared to mice presensitized to ovalbumin and challenged with ovalbumin (FIG. 2A-C). Treatment with nanocarriers (without R848) i.n. (group 9) did not reduce IL-4 levels but did reduce IL-5 and IL-13 levels compared to mice presensitized to ovalbumin and challenged with ovalbumin (FIG. 2A-C). Mice treated i.n. with nanocarriers with R848 had increased levels of IL-12p40 compared to all other groups of mice (FIG. 2D).

Together, these results indicate that treatment of mice presensitized to ovalbumin with nanocarriers containing R848 (either i.p. or i.n.) leads to decreased eosinophils in the BALF, decreased Th2 cytokines (IL-4, IL-5, and IL-13), and increased Th1 cytokines (IL-12p40). Treatment with these nanocarriers was comparable to treatment with either CpG or R848 i.p.

What is claimed is:

1. A composition for treatment of a condition comprising:
synthetic nanocarriers comprising (1) an immunofeature surface, and (2) a Th1 biasing immunostimulatory agent coupled to the synthetic nanocarriers; and
a pharmaceutically acceptable excipient;
wherein the immunofeature surface does not comprise antigen that is relevant to treatment of the condition in an amount sufficient to provoke an adaptive immune response to the antigen that is relevant to treatment of the condition.

2. The composition of claim 1, wherein the immunofeature surface comprises no antigen that is relevant to treatment of the condition.

3. The composition of claim 1, wherein the antigen that is relevant to treatment of the condition comprises an allergen.

4. The composition of claim 1, wherein the antigen that is relevant to treatment of the condition comprises a tumor antigen.

5. The composition of claim 1, wherein the antigen that is relevant to treatment of the condition comprises a chronic infectious agent antigen.

6. The composition of claim 1, wherein the immunofeature surface comprises a non-antigenic immunofeature surface.

7. The composition of claim 1, wherein the synthetic nanocarrier further comprises a T-cell antigen.

8. The composition of claim 1, wherein the synthetic nanocarriers comprise a polymeric matrix.

9. The composition of claim 1, wherein the Th1 biasing immunostimulatory agent comprises one or more of imidazoquinoline amine, imidazopyridine amine, 6,7-fused cycloalkylimidazopyridine amine, and 1,2-bridged imidazoquinoline amine, CpG, immunostimulatory RNA, lipopolysacharide, VSV-G, or HMGB-1.

10. The composition of claim 1, wherein the immunofeature surface comprises nicotine and derivatives thereof, methoxy groups, positively charged amine groups, sialyllactose, and avidin and/or avidin derivatives, and residues of any of the above.

11. The composition of claim 1, wherein the synthetic nanocarriers comprise synthetic nanocarriers that are spheres, cubes, cylinders, cones, or pyramids.

12. The composition of claim 1, wherein a minimum dimension of at least 75% of the synthetic nanocarriers in a sample, based on a total number of synthetic nanocarriers in the sample, is greater than 100 nm.

13. The composition of claim 12, wherein the antigen that is relevant to treatment of the condition comprises an allergen.

14. The composition of claim 12, wherein the antigen that is relevant to treatment of the condition comprises a tumor antigen.

15. The composition of claim 12, wherein the antigen that is relevant to treatment of the condition comprises a chronic infectious agent antigen.

16. The composition of claim 12, wherein the immunofeature surface comprises a non-antigenic immunofeature surface.

17. The composition of claim 12, wherein the synthetic nanocarriers further comprise a T-cell antigen.

18-21. (canceled)

22. A method comprising:
administering the composition of claim 1 to a subject.

23-41. (canceled)

42. A method comprising:
identifying a subject suffering from a condition;
providing a composition that comprises synthetic nanocarriers that comprise (1) an APC targeting feature, and (2) a Th1 biasing immunostimulatory agent coupled to the synthetic nanocarriers; and a pharmaceutically acceptable excipient; and
administering the composition to the subject;
wherein the administration of the composition does not further comprise co-administration of an antigen that is relevant to treatment of the condition.

43-56. (canceled)

57. A method comprising:
providing a composition comprising synthetic nanocarriers that comprise a Th1 biasing immunostimulatory agent and an APC targeting feature;
administering the composition to a subject; and
administering an antigen to the subject to which a Th1 biased response is clinically beneficial at a time different from administration of the composition to the subject;
wherein administration of the antigen comprises passive administration or active administration.

58-68. (canceled)
